(19) United States
(12) Patent Application Publication
(10) Pub. No.: US 2025/0161326 A1
(43) Pub. Date: May 22, 2025

(54) COMBINATION THERAPY

(71) Applicant: INTERCEPT PHARMACEUTICALS, INC., Morristown, NJ (US)

(72) Inventors: Antonio CIVITARESE, La Jolla, CA (US); Laurent VERNILLET, Brookfield, CT (US)

(21) Appl. No.: 18/833,630

(22) PCT Filed: Jan. 30, 2023
(86) PCT No.: PCT/US2023/011902

§ 371 Date: Jul. 26, 2024

Related U.S. Application Data
(60) Provisional application No. 63/304,394, filed on Jan. 28, 2022.

(51) Int. Cl. A61K 31/575; A61K 31/195; A61P 1/16

(52) U.S. Cl. CPC A61K 31/575; A61K 31/195; A61P 1/16

(57) ABSTRACT

The present disclosure relates to a combination of an FXR agonist and a fibrate. Also disclosed is use of the combination for the treatment, amelioration or prevention of an FXR mediated disease or condition, such as primary biliary cholangitis (PBC).

### CROSS-REFERENCE TO RELATED APPLICATIONS

This application claims priority to, and the benefit of, U.S. Provisional Application No. 63/304,394, filed Jan. 28, 2022, the contents of which are incorporated by reference in their entirety for all purposes.

### BACKGROUND

Primary biliary cholangitis (PBC) is a serious, life-threatening, cholestatic liver disease of unknown etiology that, without treatment, frequently progresses to hepatic fibrosis and eventual cirrhosis, hepatic decompensation, and necessitates liver transplantation or results in death. Subjects with advanced PBC disease are also predisposed to hepatocellular carcinoma. PBC is a rare disease with reported prevalence in the United States (US) of about 40.2/100 000. PBC disproportionately affects women more than men by approximately 10:1 and is typically diagnosed in patients between 40 and 60 years of age.

Historically, the only approved drug therapy for PBC has been the bile acid ursodeoxycholic acid (UDCA), a physiological constituent of human bile. While UDCA therapy has a marked effect on the treatment of PBC, up to 50% of patients show a suboptimal or no response to UDCA. Such patients are at significantly increased risk of a poor clinical outcome due to PBC disease progression.

Fibrates have anticholestatic, anti-inflammatory, and antifibrotic effects and have recently shown the potential to further improve the biochemical markers of PBC. The mechanisms that underlie these effects are complementary, and largely mediated through activation of peroxisome proliferator activated receptors. Fibrate treatment has been found promising in ameliorating liver biochemical tests in UDCA unresponsive patients, either as monotherapy or in combination with UDCA. Bezafibrate (BZF) has been identified as a potential anticholestatic agent for the treatment of PBC with an inadequate response to UDCA.

Obeticholic acid (OCA), a farnesoid X receptor (FXR) agonist and modified bile acid derived from the primary human bile acid chenodeoxycholic acid (CDCA), was developed for the treatment of PBC (FXR mediated disease) and to provide patients who have an inadequate response to or poor tolerance of UDCA, a novel treatment option that was safe and effective. OCA is approved for use under the tradename OCALIVA by the US Food and Drug Administration (FDA), European Medicines Agency (EMA), Health Canada, and other regulatory agencies for the treatment of PBC in combination with UDCA in adults with inadequate response to UDCA, or as monotherapy in adults unable to tolerate UDCA. However, OCA monotherapy can cause itching (pruritus) as an adverse event.

There is a need for an improved therapy for the treatment of PBC and other FXR mediated diseases, especially in patients who have an inadequate response to or cannot tolerate existing therapies. The present disclosure addresses the need.

### SUMMARY

The present disclosure relates to a method for treating or preventing an FXR mediated disease or condition comprising administering to a subject in need thereof an FXR agonist of the present disclosure, which is a compound of formula 1 (also referred to herein as OCA or obeticholic acid):

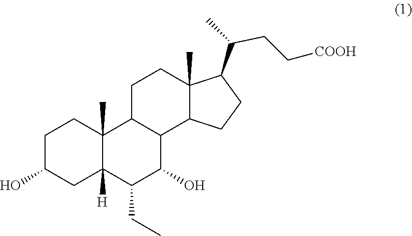 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt, solvate, or amino acid, sulfate or glucuronide conjugate, or prodrug thereof and a fibrate of the present disclosure, which is bezafibrate or a pharmaceutically acceptable salt or ester thereof.

The present disclosure relates to FXR mediated diseases or conditions in which elevated concentrations of circulating lipid compounds in the blood are involved (such as cholesterol and triglycerides), reducing the level of a liver enzyme, or inhibiting or reversing fibrosis, comprising administering a therapeutically effective amount of a pharmaceutical composition of the present invention to a subject in need thereof. In certain instances, the FXR mediated disease or condition is primary biliary cholangitis (PBC), previously known as primary biliary cirrhosis, primary sclerosing cholangitis (PSC), chronic liver disease, nonalcoholic fatty liver disease (NAFLD), nonalcoholic steatohepatitis (NASH), hepatitis C infection, alcoholic liver disease, liver damage due to progressive fibrosis, or liver fibrosis. In some instances, the FXR mediated disease or condition is portal hypertension, bile acid diarrhea, drug-induced cholestasis, hereditary cholestasis, biliary atresia, or intrahepatic cholestasis of pregnancy. In some instances, the FXR mediated disease or condition is hyperlipidemia, high LDL-cholesterol, high HDL-cholesterol, or high triglycerides.

The methods of the present disclosure address unmet needs in the treatment or prevention of an FXR mediated disease or disorder.

### BRIEF DESCRIPTION OF THE DRAWINGS

FIG. 1 is a diagram showing the study design of a double-blind treatment period.

FIG. 2 is a drug-induced liver injury (DILI) algorithm.

### DETAILED DESCRIPTION

The present disclosure relates to the treatment of FXR mediated diseases or conditions. FXR is a nuclear receptor which acts as a key regulator of cholesterol homeostasis, triglyceride synthesis and lipogenesis (Crawley, Expert Opinion Ther. Patents 2010, 20, 1047-1057). This receptor is expressed in various organs and shown to be involved in many diseases and conditions, including liver diseases, lung diseases, renal diseases, intestinal diseases, and heart diseases, and biological processes, such as glucose metabolism, insulin metabolism, and lipid metabolism.

The present disclosure is directed to a method for treating or preventing an FXR mediated disease or condition, reducing the level of a liver enzyme, or inhibiting or reversing fibrosis comprising administering to a subject in need thereof a combination of an FXR agonist of the present disclosure, which is a compound of formula 1 (also referred to herein as OCA or obeticholic acid):

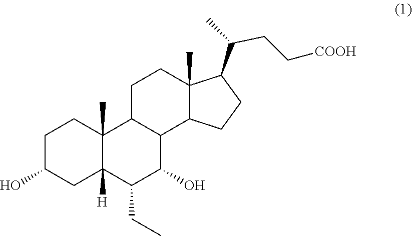 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt, solvate, or amino acid, sulfate or glucuronide conjugate, or prodrug thereof, and a fibrate of the present disclosure, which is bezafibrate (BZF) or a pharmaceutically acceptable salt or ester thereof. The present disclosure also relates to a concomitant use of OCA and BZF to improve efficacy and tolerability or safety compared to the existing treatments (e.g., the UDCA mono or combination therapies or treatment with OCA alone).

In one aspect, the amino acid conjugate of OCA is a glycine conjugate. In one aspect, the amino acid conjugate of OCA is a taurine conjugate.

The present disclosure also describes pharmaceutical compositions, packs or kits, and therapeutic uses of the combination.

One of the problems to be solved by the present disclosure is the identification of combination therapies for the treatment or prevention of conditions related to elevated concentrations of circulating lipid compounds (such as cholesterol and triglycerides) in the blood, e.g., a cholestatic liver condition such as PBC, as well as for the reduction of circulating lipid compounds (e.g., cholesterol, LDL, and triglycerides) in the blood, and for the reduction of bilirubin and/or liver enzymes, such as alkaline phosphatase (ALP, AP, or Alk Phos), alanine aminotransferase (ALT), aspartate aminotransferase (AST), gamma-glutamyl transpeptidase (GGT), lactate dehydrogenase (LDH), and 5′ nucleotidase. Although drugs for conditions related to elevated lipid levels and/or liver enzyme levels are available, these drugs are often not suitable for many patients for a variety of reasons. For example, certain drugs are ineffective for patients who have developed drug resistance, such as in the case of patients resistant to ursodeoxycholic acid. Some drugs may be inadequate for treatment when administered alone. Some drugs may require administration of high doses, or more frequent administration, due to extensive metabolism into inactive or less potent metabolites. The combination therapies described herein can solve the problems mentioned above and can have one or more advantages of, e.g., synergism, reducing the number of daily doses without compromising efficacy, lowering lipids (both cholesterol and triglycerides) in patients with PBC whose elevated lipid levels are resistant to conventional therapy, improved potency, selectivity, tissue penetration, half-life, and/or metabolic stability.

In some embodiments, the FXR mediated disease or condition is a cholestatic liver disease. In some embodiments, the disease or condition is PBC.

In one aspect, the present disclosure also relates to a method of mitigating adverse events elicited or caused by OCA monotherapy (e.g., pruritus), comprising administering the disclosed combination of about 5-10 mg OCA or a pharmaceutically acceptable salt, solvate, or amino acid, sulfate or glucuronide conjugate, or prodrug thereof and about 100 mg BZF.

In another aspect, the present disclosure also provides a method for decreasing liver enzymes, comprising administering a therapeutically effective amount of OCA or a pharmaceutically acceptable salt, solvate, or amino acid, sulfate or glucuronide conjugate, or prodrug thereof and BZF. In some embodiments, the liver enzyme is alkaline phosphatase, 7-glutamyl transpeptidase (GGT), and/or 5′ nucleotidase.

In some embodiments, the methods described herein relate to treating or preventing an FXR mediated disease or condition where a subject is not suffering from a cholestatic condition. In some embodiments, the methods described herein relate to treating or preventing an FXR mediated disease or condition where the subject is suffering from a cholestatic condition.

In certain instances, the methods described herein also include assessing, monitoring, measuring, or otherwise detecting liver function. Assessing, monitoring, measuring, or otherwise detecting liver function can be performed before, during, or after a titration period described herein, or in other instances, performed during the course of any treatment described herein. Liver function can be determined by, for example, assessing, monitoring, measuring, or otherwise detecting a level of one or more liver biomarkers compared to a control. In certain instances, the control is a baseline taken from the patient before beginning treatment. In other instances, the control is a preestablished baseline considered as a normal value. Values for measure or detection of liver function biomarkers and controls can be expressed as a comparison to Upper Limit of Normal (ULN).

In some embodiments, the methods of the present disclosure comprise a step of assessing, monitoring, measuring, or otherwise detecting liver function. In some embodiments, the step of assessing, monitoring, measuring, or otherwise detecting liver function comprises a non-invasive assay. In some embodiments, the non-invasive assay is a HepQuant SHUNT assay.

In some embodiments, the HepQuant SHUNT assay comprises measuring clearance of cholate from both the systemic circulation and portal circulation. In some embodiments, the cholate is labeled. In some embodiments, the cholate is isotopically labeled. In some embodiments, the cholate is isotopically labeled with a carbon isotope or a hydrogen isotope. In some embodiments, the cholate is isotopically labeled with ¹³C or deuterium. In some embodiments, the HepQuant SHUNT assay comprises intravenously administering (e.g., injecting) ¹³C labeled cholate. In some embodiments, the HepQuant SHUNT assay comprises orally administering deuterium labeled cholate. In some embodiments, the HepQuant SHUNT assay comprises intravenously administering ¹³C labeled cholate, and orally administering deuterium labeled cholate. In some embodiments, the HepQuant SHUNT assay comprises collecting a blood sample from the subject before the subject is administered with the cholate. In some embodiments, the HepQuant SHUNT assay comprises collecting a blood sample from the subject after cholate has been administered to the subject. In some embodiments, the HepQuant SHUNT assay comprises taking a blood sample from the subject 5, 20, 45, 60, and/or 90 minutes after administration of the cholate. In some embodiments, the HepQuant SHUNT assay comprises analyzing the blood samples to generate a Disease Severity Index (Index).

In some embodiments, the HepQuant SHUNT assay comprises:
(a) collecting a blood sample from a subject (e.g., a patient in need of treatment described herein) before the subject is administered with cholate;
(b) intravenously administering ¹³C labeled cholate, and orally administering deuterium labeled cholate, to the subject;
(c) collecting a blood sample from the subject; and
(d) analyzing the blood samples from Steps (a) and (c) to generate a Disease Severity Index.

Liver biomarkers can be used to ascertain and quantify the efficacy of the course of treatment of the present disclosure. In other instances, liver biomarkers described herein can be used to ascertain and quantify liver function during the course of treatment of the present disclosure. Liver biomarkers can also be used to predict whether a patient or patient population is susceptible to treatment described herein. In some embodiments, the liver biomarkers include assessing, monitoring, measuring or otherwise detecting an amount or level of aspartate transaminase (AST), alanine transaminase (ALT), alkaline phosphatase (ALP), bilirubin, glycine conjugated obeticholic acid, taurine conjugated obeticholic acid, a bile acid, a bile acid glycine conjugate, or a bile acid taurine conjugate. For example, the liver biomarker assessed, monitored, measured, or detected can be ALP.

The ALP level can be a measure of ULN. In some embodiments, a patient before treatment can have an ALP level of at least 1.1× ULN to at least 20× ULN; at least 1.1× ULN to at least 15× ULN; at least 1.1× ULN to at least 12× ULN; at least 1.1× ULN to at least 10× ULN; at least 1.1× ULN to at least 8× ULN; at least 1.1× ULN to at least 6× ULN; at least 1.1× ULN to at least 5× ULN; at least 1.1× ULN to at least 4× ULN; at least 1.1× ULN to at least 3× ULN; or at least 1.1× ULN to at least 2× ULN.

A patient before a treatment described herein can have an ALP level of about 1.5× ULN to about 20× ULN; about 1.5× ULN to about 15× ULN; about 1.5× ULN to about 10 ULN; about 1.5× ULN to about 5× ULN; or about 1.5× ULN to about 3× ULN. A patient before treatment can have an ALP level of about 1.5×, 2×, 3×, 4×, 5×, 8×, 10×, 15×, or 20× ULN.

A patient before a treatment described herein can have an ALP level of greater than about 1.5×, 2×, 3×, 4×, 5×, 8×, 10×, 15×, or 20× ULN. In some embodiments, a patient has an ALP level of about 1.5× ULN. In some embodiments, a patient has an ALP level of about 2× ULN. In some embodiments, a patient has a ALP level of about 5× ULN. In some embodiments, a patient has an ALP level of about 10× ULN. In some embodiments, a patient has an ALP level of about 15× ULN. In some embodiments, a patient has an ALP level greater than about 1.5× ULN. In some embodiments, a patient has an ALP level greater than about 2× ULN. In some embodiments, a patient has a ALP level greater than about 5× ULN. In some embodiments, a patient has an ALP level greater than about 10× ULN. In some embodiments, a patient has an ALP level greater than about 15× ULN.

In another example, the liver biomarker assessed, monitored, measured, or detected can be bilirubin. The bilirubin level can be a measure of ULN. In some embodiments, a patient before treatment can have a bilirubin level of at least 1.1× ULN to at least 20× ULN; at least 1.1× ULN to at least 15× ULN; at least 1.1× ULN to at least 12× ULN; at least 1.1× ULN to at least 10× ULN; at least 1.1× ULN to at least 8× ULN; at least 1.1× ULN to at least 6× ULN; at least 1.1× ULN to at least 5× ULN; at least 1.1× ULN to at least 4× ULN; at least 1.1× ULN to at least 3× ULN; or at least 1.1× ULN to at least 2× ULN.

A patient before a treatment described herein can have a bilirubin level of about 1.5× ULN to about 20× ULN; about 1.5× ULN to about 15× ULN; about 1.5× ULN to about 10 ULN; about 1.5× ULN to about 5× ULN; or about 1.5× ULN to about 3× ULN. In another example a patient before a treatment described herein can have a bilirubin level of about 2× ULN to about 20× ULN; about 2× ULN to about 15× ULN; about 2× ULN to about 10 ULN; about 2× ULN to about 5× ULN; or about 2× ULN to about 3× ULN. In another example a patient before a treatment described herein can have a bilirubin level of greater than about 2× ULN to greater than about 20× ULN; greater than about 2× ULN to greater than about 15× ULN; greater than about 2× ULN to greater than about 10 ULN; greater than about 2× ULN to greater than about 5× ULN; or greater than about 2× ULN to greater than about 3× ULN.

A patient before a treatment described herein can have a bilirubin level of about 1.5×, 2×, 3×, 4×, 5×, 8×, 10×, 15×, or 20× ULN. A patient before treatment can have a bilirubin level of greater than about 1.5×, 2×, 3×, 4×, 5×, 8×, 10×, 15×, or 20× ULN. In some embodiments, a patient has a bilirubin level greater than about 2× ULN. In some embodiments, a patient has a bilirubin level greater than about 5× ULN. In some embodiments, a patient has a bilirubin level greater than about 10× ULN. In some embodiments, a patient has a bilirubin level greater than about 15× ULN. In some embodiments, a patient has a bilirubin level less than about 2× ULN. In some embodiments, a patient has a bilirubin level less than about 5× ULN. In some embodiments, a patient has a bilirubin level less than about 10× ULN. In some embodiments, a patient has a bilirubin level less than about 15× ULN.

In some instances, it can be useful to assess, monitor, measure, or detect ALP and bilirubin to assess, monitor, measure, or otherwise detect liver function or changes in liver function during treatment described herein. In certain instances, a patient has an ALP level as provided above (e.g., about 1.5× ULN to about 10× ULN) and a bilirubin level as provided above (e.g., less than about 5× ULN). In some embodiments, the patient has an ALP level between about 1.5× ULN to about 10× ULN and a bilirubin level less than about 2× ULN.

Treatment described herein can reduce the levels of ALP and/or bilirubin in a patient described herein. For example, treatment of an FXR mediated disease or condition described herein can reduce the level of ALP by 2, 4, 5, 6, 8, 9, 10, 12, 15, 18, 20, 21, 22, 23, 24, 25, 26, 27, 28, 29, 30, 35, 40, 45, 50, 55, 60, 65, 70, 75, 80, 85, 88, 90, 92, 94, 96, 97, 98, 99, 99.2, 99.4, 99.6, 99.7, 99.8, 99.9, or 100%. In another example, the level of ALP can be reduced by at least 100%, at least 125%, at least 150%, at least 175%, at least 200%, at least 225%, at least 250% or at least 300%. In some embodiments, the FXR mediated disease or condition is PBC.

In another example, the level of ALP can be reduced by about 5% to about 50%; about 10% to about 55%; about 10% to about 45%; about 10% to about 40%; about 10% to about 33%, about 10% to about 30%; about 15% to about 30%; about 15% to about 25%; about 20% to about 50%, about 20% to about 40%; about 20% to about 35%; about 20% to about 30%; 20% to about 27%; or about 20% to about 27%. In another example, the level of ALP can be reduced by at least 50%. The level of ALP can be reduced by at least 40%. The level of ALP can be reduced by at least 35%. The level of ALP can be reduced by at least 30%. The level of ALP can be reduced by at least 27%. The level of ALP can be reduced by at least 25%. The level of ALP can be reduced by at least 20%.

The reduction of ALP levels can be represented by the fold change over ULN. For example, treatment described herein can reduce the ALP level of a patient described herein to less than about 5× ULN; less than about 4× ULN, less than about 3× ULN, less than about 2× ULN, less than about 1.7× ULN, less than about 1.5× ULN, less than about 1.25× ULN, or less than about ULN.

In another example, the ALP level is reduced by at least 1, 2, 3, 4, 5, 6, 7, 8, 9, 10, 15, 20, 25, 30, 40, or 5-fold compared to a baseline value. For example, the ALP level after treatment described herein can be reduced by 1, 1.2, 1.4, 1.6, 1.8, or 2-fold, including intervening values therein, compared to a baseline value. In another example, the ALP level can be reduced by 2, 2.2, 2.4, 2.6, 2.8, or 3-fold, including intervening values therein, compared to a baseline value. In another example, the ALP level can be reduced 3, 4, or 5-fold, including intervening values therein, compared to a baseline value. In another example, the ALP level can be reduced 5, 7, 9, or 10-fold, including intervening values therein, compared to a baseline value. In another example, the ALP level can be reduced 10, 12, 15,or 20-fold, including intervening values therein, compared to a baseline value.

Treatment of a disease or condition described herein can reduce the level of bilirubin by 2, 4, 5, 6, 8, 9, 10, 12, 15, 18, 20, 21, 22, 23, 24, 25, 26, 27, 28, 29, 30, 35, 40, 45, 50, 55, 60, 65, 70, 75, 80, 85, 88, 90, 92, 94, 96, 97, 98, 99, 99.2, 99.4, 99.6, 99.7, 99.8, 99.9, or 100%. In another example, the level of bilirubin can be reduced by at least 100%, at least 125%, at least 150%, at least 175%, at least 200%, at least 225%, at least 250% or at least 300%.

In another example, the level of bilirubin can be reduced by about 5% to about 50%; about 10% to about 55%; about 10% to about 45%; about 10% to about 40%; about 10% to about 33%, about 10% to about 30%; about 15% to about 30%; about 15% to about 25%; about 20% to about 50%, about 20% to about 40%; about 20% to about 35%; about 20% to about 30%; 20% to about 27%; or about 20% to about 27%. In another example, the level of bilirubin can be reduced by at least 50%. The level of bilirubin can be reduced by at least 40%. The level of bilirubin can be reduced by at least 35%. The level of bilirubin can be reduced by at least 30%. The level of bilirubin can be reduced by at least 27%. The level of bilirubin can be reduced by at least 25%. The level of bilirubin can be reduced by at least 20%.

The reduction of bilirubin levels can be represented by the fold change over ULN. For example, treatment described herein can reduce the bilirubin level of a patient described herein to less than about 5× ULN; less than about 4× ULN, less than about 3× ULN, less than about 2× ULN, less than about 1.7× ULN, less than about 1.5× ULN, less than about 1.25× ULN, or less than about ULN.

In another example, the bilirubin level is reduced by at least 1, 2, 3, 4, 5, 6, 7, 8, 9, 10, 15, 20, 25, 30, 40, or 50-fold compared to a baseline value. For example, the bilirubin level after treatment described herein can be reduced by 1, 1.2, 1.4, 1.6, 1.8, or 2-fold, including intervening values therein, compared to a baseline value. In another example, the bilirubin level can be reduced by 2, 2.2, 2.4, 2.6, 2.8, or 3-fold, including intervening values therein, compared to a baseline value. In another example, the bilirubin level can be reduced 3, 4, or 5-fold, including intervening values therein, compared to a baseline value. In another example, the bilirubin level can be reduced 5, 7, 9, or 10-fold, including intervening values therein, compared to a baseline value. In another example, the bilirubin level can be reduced 10, 12, 15, or 20-fold, including intervening values therein, compared to a baseline value.

In another embodiment, one or more biomarkers can stratify a patient population undergoing treatment described herein. For example, a PBC patient can be stratified for the risk of hepatocellular carcinoma (HCC).

In another embodiment, liver biomarkers useful for detection can include metabolites and bile acids. For example, assessing, monitoring, measuring, or otherwise detecting levels of glycine and taurine conjugates of obeticholic acid can be useful for measuring efficacy of a treatment regimen described herein. For example, assessing, monitoring, measuring, or otherwise detecting levels or detecting plasma levels of bile acids including cholic acid, chenodeoxycholic acid, deoxycholic acid, lithocholic acid, and ursodeoxycholic acid, including glycine and taurine conjugates thereof, and optionally comparing the levels to a control, can be useful for measuring efficacy of a treatment regimen described herein.

In still other embodiments, calculating an AST to platelet index (APRI) can be useful for assessing, monitoring, measuring, or otherwise detecting liver function (including changes therein). The methods described herein can reduce the APRI of a patient described herein. In certain instances, monitoring or measuring the APRI can be used to determine efficacy of the treatment described herein. In some embodiments, a reduction in APRI is observed in a patient (e.g., a PBC patient) after treatment described herein. For example, the APRI may be reduced by about 5% to about 50% in patients treated according to the methods of the present disclosure relative to baseline levels measured before dose administration. The reduction may be up to about 5%, 10%, 15%, 20%, 25%, 30%, 35%, 40%, 45% or 50%.

The present disclosure relates to a method for treating an FXR mediated disease or condition in a patient in need thereof, the method comprising: (1) administering to the patient about 5-10 mg OCA or a pharmaceutically acceptable salt, solvate, or amino acid, sulfate or glucuronide conjugate, or prodrug thereof and about 100 mg BZF; (2) assessing liver function (optionally before, during, and after said titration period) of the patient by: (a) calculating an AST to platelet ratio (APRI) score for the patient; (b) measuring the level of one or more liver biomarkers selected from ALP, bilirubin, AST, ALT, glycine conjugated obeticholic acid, taurine conjugated obeticholic acid, a bile acid, a bile acid glycine conjugate, or a bile acid taurine conjugate; or (c) a HepQuant SHUNT assay described herein; (3) wherein a reduced APRI score compared to a control or a reduced level of the one or more liver biomarkers compared to a control indicates non-impaired liver function; (4) assessing tolerance of the patient to the starting dose by grading the severity of one or more adverse effects, if present; and (5) administering an adjusted dose of the composition (if necessary) (wherein the adjusted dose comprises an amount equal to or greater than an amount of the starting dose). In some embodiments, the FXR mediated disease or condition is PBC.

The present disclosure relates to a method of treating an FXR mediated disease or condition in a patient in need thereof, comprising administering to the patient about 5-10 mg OCA or a pharmaceutically acceptable salt, solvate, or amino acid, sulfate or glucuronide conjugate, or prodrug thereof and about 100 mg BZF (optionally in a titration period) wherein
the liver function of the patient is assessed (optionally before, during, and after said titration period) by calculating an AST to platelet ratio (APRI) score for said patient or by measuring the level of one or more liver biomarkers selected from ALP, bilirubin, AST, ALT, glycine conjugated obeticholic acid, taurine conjugated obeticholic acid, a bile acid, a bile acid glycine conjugate, or a bile acid taurine conjugate, wherein a reduced APRI score compared to a control or a reduced level of said one or more liver biomarkers compared to a control indicates non-impaired liver function; and
the tolerance of the patient to said starting dose is assessed by grading the severity of one or more adverse effects, if present; and the combination is prepared to be administered as an adjusted dose (wherein said adjusted dose comprises an amount equal to or greater than an amount of said starting dose). In some of the embodiments, the FXR mediated disease or condition is PBC.

The present disclosure relates to a method for treating an FXR mediated disease or condition in a patient in need thereof, the method comprising administering a combination of OCA or a pharmaceutically acceptable salt, solvate, or amino acid, sulfate or glucuronide conjugate, or prodrug thereof in the amount of about 5-10 mg and bezafibrate in the amount of about 100 mg, wherein the combination is administered once daily (QD).

The present disclosure relates to a method for treating an FXR mediated disease or condition in a patient in need thereof, the method comprising administering a combination comprising OCA or a pharmaceutically acceptable salt, solvate, or amino acid, sulfate or glucuronide conjugate, or prodrug thereof in the amount of 5 mg and bezafibrate in the amount of 100 mg, wherein the combination is administered QD.

The present disclosure relates to a method for treating an FXR mediated disease or condition in a patient in need thereof, the method comprising administering a combination comprising OCA or a pharmaceutically acceptable salt, solvate, or amino acid, sulfate or glucuronide conjugate, or prodrug thereof in the amount of 10 mg and bezafibrate in the amount of 100 mg, wherein the combination is administered QD.

The present disclosure relates to a combination comprising OCA or a pharmaceutically acceptable salt, solvate, or amino acid, sulfate or glucuronide conjugate, or prodrug thereof in the amount of about 5-10 mg and bezafibrate in the amount of about 100 mg for use in the treatment of an FXR mediated disease or condition, wherein the combination is for administration once daily.

The present disclosure relates to a combination comprising OCA or a pharmaceutically acceptable salt, solvate, or amino acid, sulfate or glucuronide conjugate, or prodrug thereof in the amount of 5 mg and bezafibrate in the amount of 100 mg for use in the treatment of an FXR mediated disease or condition, wherein the combination is for administration once daily.

The present disclosure relates to a combination comprising OCA or a pharmaceutically acceptable salt, solvate, or amino acid, sulfate or glucuronide conjugate, or prodrug thereof in the amount of 10 mg and bezafibrate in the amount of 100 mg for use in the treatment of an FXR mediated disease or condition, wherein the combination is for administration once daily.

The present disclosure relates to use of a combination comprising OCA or a pharmaceutically acceptable salt, solvate, or amino acid, sulfate or glucuronide conjugate, or prodrug thereof in the amount of about 5-10 mg and bezafibrate in the amount of about 100 mg in the manufacture of a medicament for the treatment of an FXR mediated disease or condition, wherein the combination is for administration once daily.

The present disclosure relates to use of a combination comprising OCA or a pharmaceutically acceptable salt, solvate, or amino acid, sulfate or glucuronide conjugate, or prodrug thereof in the amount of 5 mg and bezafibrate in the amount of 100 mg in the manufacture of a medicament for the treatment of an FXR mediated disease or condition, wherein the combination is for administration once daily.

The present disclosure relates to use of a combination comprising OCA or a pharmaceutically acceptable salt, solvate, or amino acid, sulfate or glucuronide conjugate, or prodrug thereof in the amount of 10 mg and bezafibrate in the amount of 100 mg in the manufacture of a medicament for the treatment of an FXR mediated disease or condition, wherein the combination is for administration once daily.

The present disclosure relates to a method for treating an FXR mediated disease or condition in a patient in need thereof, the method comprising administering to the patient OCA or a pharmaceutically acceptable salt, solvate, or amino acid, sulfate or glucuronide conjugate, or prodrug thereof in the amount of about 5-10 mg QD and bezafibrate in the amount of about 100 mg QD.

The present disclosure relates to a method for treating an FXR mediated disease or condition in a patient in need thereof, the method comprising administering to the patient OCA or a pharmaceutically acceptable salt, solvate, or amino acid, sulfate or glucuronide conjugate, or prodrug thereof in the amount of 5 mg QD and bezafibrate in the amount of 100 mg QD.

The present disclosure relates to a method for treating an FXR mediated disease or condition in a patient in need thereof, the method comprising administering to the patient OCA or a pharmaceutically acceptable salt, solvate, or amino acid, sulfate or glucuronide conjugate, or prodrug thereof in the amount of 10 mg QD and bezafibrate in the amount of 100 mg QD.

The present disclosure relates to OCA or a pharmaceutically acceptable salt, solvate, or amino acid, sulfate or glucuronide conjugate, or prodrug thereof for use in combination with bezafibrate in the treatment of an FXR mediated disease or condition, wherein OCA or a pharmaceutically acceptable salt, solvate, or amino acid, sulfate or glucuronide conjugate, or prodrug thereof is for administration in the amount of about 5-10 mg QD and bezafibrate is for administration in the amount of about 100 mg QD.

The present disclosure relates to OCA or a pharmaceutically acceptable salt, solvate, or amino acid, sulfate or glucuronide conjugate, or prodrug thereof for use in combination with bezafibrate in the treatment of an FXR mediated disease or condition, wherein OCA or a pharmaceutically acceptable salt, solvate, or amino acid, sulfate or glucuronide conjugate, or prodrug thereof is for administration in the amount of 5 mg QD and bezafibrate is for administration in the amount of 100 mg QD.

The present disclosure relates to OCA or a pharmaceutically acceptable salt, solvate, or amino acid, sulfate or glucuronide conjugate, or prodrug thereof for use in combination with bezafibrate in the treatment of an FXR mediated disease or condition, wherein OCA or a pharmaceutically acceptable salt, solvate, or amino acid, sulfate or glucuronide conjugate, or prodrug thereof is for administration in the amount of 10 mg QD and bezafibrate is for administration in the amount of 100 mg QD.

The present disclosure relates to use of OCA or a pharmaceutically acceptable salt, solvate, or amino acid, sulfate or glucuronide conjugate, or prodrug thereof in combination with bezafibrate in the manufacture of a medicament for use in the treatment of an FXR mediated disease or condition, wherein OCA or a pharmaceutically acceptable salt, solvate, or amino acid, sulfate or glucuronide conjugate, or prodrug thereof is for administration in the amount of about 5-10 mg QD and bezafibrate is for administration in the amount of about 100 mg QD.

The present disclosure relates to use of OCA or a pharmaceutically acceptable salt, solvate, or amino acid, sulfate or glucuronide conjugate, or prodrug thereof in combination with bezafibrate in the manufacture of a medicament for use in the treatment of an FXR mediated disease or condition, wherein OCA or a pharmaceutically acceptable salt, solvate, or amino acid, sulfate or glucuronide conjugate, or prodrug thereof is for administration in the amount of 5 mg QD and bezafibrate is for administration in the amount of 100 mg QD.

The present disclosure relates to use of OCA or a pharmaceutically acceptable salt, solvate, or amino acid, sulfate or glucuronide conjugate, or prodrug thereof in combination with bezafibrate in the manufacture of a medicament for use in the treatment of an FXR mediated disease or condition, wherein OCA or a pharmaceutically acceptable salt, solvate, or amino acid, sulfate or glucuronide conjugate, or prodrug thereof is for administration in the amount of 10 mg QD and bezafibrate is for administration in the amount of 100 mg QD.

The present disclosure relates to a combinational therapy for the treatment of an FXR mediated disease or condition, comprising administration of OCA or a pharmaceutically acceptable salt, solvate, or amino acid, sulfate or glucuronide conjugate, or prodrug thereof in the amount of about 5-10 mg QD and bezafibrate in the amount of about 100 mg QD.

The present disclosure relates to a combinational therapy for the treatment of an FXR mediated disease or condition, comprising administration of OCA or a pharmaceutically acceptable salt, solvate, or amino acid, sulfate or glucuronide conjugate, or prodrug thereof in the amount of 5 mg QD and bezafibrate in the amount of 100 mg QD.

The present disclosure relates to a combinational therapy for the treatment of an FXR mediated disease or condition, comprising administration of OCA or a pharmaceutically acceptable salt, solvate, or amino acid, sulfate or glucuronide conjugate, or prodrug thereof in the amount of 10 mg QD and bezafibrate in the amount of 100 mg QD.

In some embodiments, the methods, combinations for use, uses, and combination therapies of the present disclosure comprise administering or administration for a period of at least 4 weeks. In some embodiments, the methods, combinations for use, uses, and combination therapies of the present disclosure comprise administering or administration for a period of at least 12 weeks. In some embodiments, the methods, combinations for use, uses, and combination therapies of the present disclosure comprise administering or administration for a period of 1-12 weeks. In some embodiments, the methods, combinations for use, uses, and combination therapies of the present disclosure comprise administering or administration for a period of 12-48 weeks.

In some embodiments, OCA or a pharmaceutically acceptable salt, solvate, or amino acid, sulfate or glucuronide conjugate, or prodrug thereof is in a tablet form.

In some embodiments, bezafibrate is in an immediate release form (e.g., immediate release tablet). In some embodiments, bezafibrate is in a sustained release form (e.g., sustained release tablet).

In some embodiments, the methods, combinations for use, uses, and combination therapies of the present disclosure further comprise a step of assessing, monitoring, measuring, or otherwise detecting liver function, as described herein (e.g., HepQuant SHUNT assay).

Also provided herein are methods to reduce or eliminate rejection failure of a liver transplant by administering the treatment described herein. In certain instances, administration of the combination described herein reduces expression or levels of ALP and/or bilirubin. In some embodiments, administration of the combination described herein reduces ALP and bilirubin levels, thereby reducing transplant complications or rejection. In another embodiment, administration of an effective amount of the combination described herein increases post-transplantation survival rate of a liver transplantee.

In the compositions, packs or kits, methods and uses of the present disclosure, OCA may be in a free form (e.g., acid) or it may be a pharmaceutically acceptable salt, solvate, or amino acid, sulfate or glucuronide conjugate, or prodrug thereof. In some embodiments, OCA is in the form of a pharmaceutically acceptable salt or amino acid conjugate (e.g., glycine or taurine conjugate).

In some embodiments, the FXR agonist of the present disclosure is the glycine conjugate of OCA, and the fibrate of the present disclosure is BZF or a pharmaceutically acceptable salt or ester thereof.

In some embodiments, the FXR agonist of the present disclosure is the taurine conjugate of OCA, and the fibrate of the present disclosure is BZF or a pharmaceutically acceptable salt or ester thereof.

In some embodiments, the FXR agonist of the present disclosure is OCA or a pharmaceutically acceptable salt or amino acid conjugate thereof, and the fibrate of the present disclosure is BZF.

The disclosure also encompasses isotopically-labeled OCA or a pharmaceutically acceptable salt, solvate, or amino acid, sulfate or glucuronide conjugate, or prodrug thereof, which has a structure that is identical to that of the FXR agonist of the present disclosure of the present disclosure except that one or more atoms is replaced by an atom having an atomic mass or mass number different from the atomic mass or mass number most commonly found in nature. Examples of isotopes that can be incorporated into the FXR agonist of the present disclosure or a pharmaceutically acceptable salt, solvate, or amino acid, sulfate or glucuronide conjugate, or prodrug thereof, include isotopes of hydrogen, carbon, nitrogen, fluorine, such as ³H, ¹¹C, ¹⁴C and ¹⁸F.

OCA or a pharmaceutically acceptable salt, solvate, or amino acid, sulfate or glucuronide conjugate, or prodrug thereof that contains the aforementioned isotopes and/or other isotopes of other atoms is within the scope of the present disclosure. Isotopically labeled OCA or a pharmaceutically acceptable salt, solvate, or amino acid, sulfate or glucuronide conjugate, or prodrug thereof, for example, OCA into which a radioactive isotope(s) such as ³H and/or ¹⁴C are incorporated, are useful in drug and/or substrate tissue distribution assays. Tritiated, i.e., ³H, and carbon-14, i.e., ¹⁴C, isotopes are used for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be used in some circumstances. Isotopically labeled OCA or a pharmaceutically acceptable salt, solvate, or amino acid, sulfate or glucuronide conjugate, or prodrug thereof can be prepared by carrying out the procedures disclosed in the Schemes and/or in the Examples of the present disclosure, and substituting a readily available isotopically labeled reagent for a non-isotopically labeled reagent.

The present disclosure also provides a method for treating or preventing a disease or condition, comprising administering a therapeutically effective amount of a pharmaceutical combination of the present disclosure to a subject in need thereof. In some embodiments, the disease or condition is an FXR mediated disease or condition. Examples of the FXR mediated diseases or conditions include, but are not limited to, primary biliary cholangitis (PBC), primary sclerosing cholangitis (PSC), portal hypertension, bile acid diarrhea, a chronic liver disease, nonalcoholic fatty liver disease (NAFLD), nonalcoholic steatohepatitis (NASH), hepatitis C infection, an alcoholic liver disease, liver damage due to progressive fibrosis, liver fibrosis, drug-induced cholestasis, hereditary cholestasis, biliary atresia, and intrahepatic cholestasis of pregnancy. In some embodiments, the disease or condition is chronic liver disease. In some embodiments, the disease or condition is a cholestatic liver disease. In some embodiments, the disease or condition is PBC. In some embodiments, the disease or condition is NASH. In some embodiments, the disease or condition is liver fibrosis. In some embodiments, the disease or condition is liver fibrosis associated with NASH.

The present disclosure also provides a method of mitigating adverse events elicited or caused by OCA monotherapy (e.g., pruritus), comprising administering the disclosed combination of OCA and BZF.

The present disclosure also provides a method for inhibiting or reversing fibrosis associated with a disease or condition described herein, comprising administering a therapeutically effective amount of a combination of the present disclosure to a subject in need thereof. In some embodiments, the fibrosis to be inhibited or reversed occurs in an organ where FXR is expressed. In some embodiments, the subject is suffering from a cholestatic condition. In some embodiments, the subject is not suffering from a cholestatic condition.

In some embodiments, a cholestatic condition is defined as having an abnormally elevated serum level of alkaline phosphatase, γ-glutamyl transpeptidase (GGT), and/or 5′ nucleotidase. In another embodiment, a cholestatic condition is further defined as presenting with at least one clinical symptom. In some embodiments, the symptom is itching (pruritus). In another embodiment, a cholestatic condition is selected from the group consisting of primary biliary cholangitis (PBC), primary sclerosing cholangitis (PSC), drug-induced cholestasis, hereditary cholestasis, biliary atresia, and intrahepatic cholestasis of pregnancy.

The present disclosure also provides a method for reducing lipid levels (i.e., amount of lipid), such as in the blood, comprising administering a therapeutically effective amount of a combination of the present disclosure to a subject in need thereof. In some embodiments, the method of the present disclosure reduces the lipid levels by at least 10%, 20%, 30%, 40%, 50%, 60%, 70%, 80%, or 90%, as compared to a control subject (e.g., a subject not treated according to the methods of the present disclosure). In some embodiments, the subject has elevated levels of lipid, as compared to a healthy subject (e.g., an individual without a disease or condition, such as those described herein). In some embodiments, the method of the present disclosure reduces the levels of lipid to normal levels (e.g., similar to the lipid levels in an individual without a disease or condition, such as those described herein).

In some embodiments, the lipid is cholesterol. In some embodiments, the method of the present disclosure reduces cholesterol levels by at least 10%, 20%, 30%, 40%, 50%, 60%, 70%, 80%, or 90%, as compared to a control subject (e.g., a subject not treated according to the methods of the present disclosure). In some embodiments, the subject has elevated levels of cholesterol, as compared to a healthy subject (e.g., an individual without a disease or condition, such as those described herein). In some embodiments, the method of the present disclosure reduces cholesterol levels below 400 mg/L, 350 mg/L, 300 mg/L, 250 mg/L, 240 mg/L, 230 mg/L, 220 mg/L, 210 mg/L, 200 mg/L, 190 mg/L, 180 mg/L, 170 mg/L, 160 mg/L, or 150 mg/L. In some embodiments, the method of the present disclosure reduces cholesterol levels below 200 mg/L, 190 mg/L, 180 mg/L, 170 mg/L, 160 mg/L, or 150 mg/L.

In some embodiments, the cholesterol is LDL. In some embodiments, the method of the present disclosure reduces LDL levels by at least 10%, 20%, 30%, 40%, 50%, 60%, 70%, 80%, or 90%, as compared to a control subject (e.g., a subject not treated according to the methods of the present disclosure). In some embodiments, the subject has elevated levels of LDL, as compared to a healthy subject (e.g., an individual without a disease or condition, such as those described herein). In some embodiments, the method of the present disclosure reduces LDL levels below 300 mg/L, 200 mg/L, 190 mg/L, 180 mg/L, 170 mg/L, 160 mg/L, 150 mg/L, 140 mg/L, 130 mg/L, 120 mg/L, 110 mg/L, 100 mg/L, 90 mg/L, 80 mg/L, 70 mg/L, 60 mg/L, or 50 mg/L. In some embodiments, the method of the present disclosure reduces LDL levels below 160 mg/L, 150 mg/L, 140 mg/L, 130 mg/L, 120 mg/L, 110 mg/L, 100 mg/L, 90 mg/L, 80 mg/L, 70 mg/L, 60 mg/L, or 50 mg/L. In some embodiments, the method of the present disclosure reduces LDL levels below 130 mg/L, 120 mg/L, 110 mg/L, 100 mg/L, 90 mg/L, 80 mg/L, 70 mg/L, 60 mg/L, or 50 mg/L. In some embodiments, the method of the present disclosure reduces LDL levels below 100 mg/L, 90 mg/L, 80 mg/L, 70 mg/L, 60 mg/L, or 50 mg/L. In some embodiments, the method of the present disclosure reduces LDL levels below 70 mg/L, 60 mg/L, or 50 mg/L.

In some embodiments, the lipid is triglyceride. In some embodiments, the method of the present disclosure reduces triglyceride levels by at least 10%, 20%, 30%, 40%, 50%, 60%, 70%, 80%, or 90%, as compared to a control subject (e.g., a subject not treated according to the methods of the present disclosure). In some embodiments, the subject has elevated levels of triglyceride, as compared to a healthy subject (e.g., an individual without a disease or condition, such as those described herein). In some embodiments, the method of the present disclosure reduces triglyceride levels below 800 mg/L, 700 mg/L, 600 mg/L, 500 mg/L, 400 mg/L, 300 mg/L, 200 mg/L, 190 mg/L, 180 mg/L, 170 mg/L, 160 mg/L, 150 mg/L, 140 mg/L, 130 mg/L, 120 mg/L, 110 mg/L, or 100 mg/L. In some embodiments, the method of the present disclosure reduces triglyceride levels below 200 mg/L, 190 mg/L, 180 mg/L, 170 mg/L, 160 mg/L, 150 mg/L, 140 mg/L, 130 mg/L, 120 mg/L, 110 mg/L, or 100 mg/L. In some embodiments, the method of the present disclosure reduces triglyceride levels below 150 mg/L, 140 mg/L, 130 mg/L, 120 mg/L, 110 mg/L, or 100 mg/L.

The present disclosure also provides a method for reducing the amount of bilirubin, and/or one or more liver enzymes, comprising administering a therapeutically effective amount of a combination of the present disclosure to a subject in need thereof.

In some embodiments, the method of the present disclosure reduces the amount of bilirubin by at least 10%, 20%, 30%, 40%, 50%, 60%, 70%, 80%, or 90%, as compared to a control subject (e.g., a subject not treated according to the methods of the present disclosure). In some embodiments, the subject has an elevated level of bilirubin, as compared to a healthy subject (e.g., an individual without a disease or condition, such as those described herein). In some embodiments, the method of the present disclosure reduces the level of bilirubin to a normal level (e.g., similar to the level of bilirubin in an individual without a disease or condition, such as those described herein). In a further embodiment, the method of the present disclosure reduces the level of bilirubin below 10 mg/L, 9 mg/L, 8 mg/L, 7 mg/L, 6 mg/L, 5 mg/L, 4 mg/L, 3 mg/L, 2 mg/L, 1.5 mg/L, 1.2 mg/L, or 1 mg/L. In a further embodiment, the method of the present disclosure reduces the level of bilirubin below 2 mg/L, 1.5 mg/L, 1.2 mg/L, or 1 mg/L.

In some embodiments, the liver enzyme is selected from the group consisting of alkaline phosphatase (ALP, AP, or Alk Phos), alanine aminotransferase (ALT), aspartate aminotransferase (AST), gamma-glutamyl transpeptidase (GGT), lactate dehydrogenase (LDH), and 5′ nucleotidase. In some embodiments, the method of the present disclosure reduces the amount of one or more liver enzymes by at least 10%, 20%, 30%, 40%, 50%, 60%, 70%, 80%, or 90%, as compared to a control subject (e.g., a subject not treated according to the methods of the present disclosure). In some embodiments, the subject has elevated levels of one or more liver enzymes, as compared to a healthy subject (e.g., an individual without a disease or condition, such as those described herein). In some embodiments, the method of the present disclosure reduces the levels of one or more liver enzymes (e.g., ALP, ALT, AST, GGT, LDH, and 5′ nucleotidase) to normal levels (e.g., similar to the levels of liver enzymes in an individual without a disease or condition, such as those described herein).

In a further embodiment, the method of the present disclosure reduces the level of ALP below 500 IU/L (international units per liter), 400 IU/L, 300 IU/L, 200 IU/L, 180 IU/L, 160 IU/L, or 150 IU/L. In a further embodiment, the method of the present disclosure reduces the level of ALP to from about 40 IU/L to about 150 IU/L.

In a further embodiment, the method of the present disclosure reduces the level of ALT below 200 IU/L (international units per liter), 150 IU/L, 100 IU/L, 80 IU/L, 60 IU/L, or 50 IU/L. In a further embodiment, the method of the present disclosure reduces the level of ALT to from about 5 IU/L to about 50 IU/L.

In a further embodiment, the method of the present disclosure reduces the level of AST below 200 IU/L (international units per liter), 150 IU/L, 100 IU/L, 80 IU/L, 60 IU/L, 50 IU/L, or 40 IU/L. In a further embodiment, the method of the present disclosure reduces the level of AST to from about 10 IU/L to about 50 IU/L.

In a further embodiment, the method of the present disclosure reduces the level of GGT below 200 IU/L (international units per liter), 150 IU/L, 100 IU/L, 90 IU/L, 80 IU/L, 70 IU/L, or 60 IU/L. In a further embodiment, the method of the present disclosure reduces the level of GGT to from about 15 IU/L to about 50 IU/L or from about 5 IU/L to about 30 IU/L.

In a further embodiment, the method of the present disclosure reduces the level of LDH below 500 IU/L (international units per liter), 400 IU/L, 300 IU/L, 200 IU/L, 180 IU/L, 160 IU/L, 150 IU/L, 140 IU/L, or 130 IU/L. In a further embodiment, the method of the present disclosure reduces the level of LDH to from about 120 IU/L to about 220 IU/L.

In a further embodiment, the method of the present disclosure reduces the level of 5′ nucleotidase below 50 IU/L (international units per liter), 40 IU/L, 30 IU/L, 20 IU/L, 18 IU/L, 17 IU/L, 16 IU/L, 15 IU/L, 14 IU/L, 13 IU/L, 12 IU/L, 11 IU/L, 10 IU/L, 9 IU/L, 8 IU/L, 7 IU/L, 6 IU/L, or 5 IU/L. In a further embodiment, the method of the present disclosure reduces the level of 5′ nucleotidase to from about 2 IU/L to about 15 IU/L.

In some embodiments, the methods of the present disclosure comprise administering to a subject in need thereof an effective amount of OCA, in combination with BZF, and optionally one or more pharmaceutically acceptable carriers. In a further embodiment, the method comprises administering to a subject in need thereof an effective amount of a compound of OCA or a pharmaceutically acceptable salt, solvate, or amino acid, sulfate or glucuronide conjugate, or prodrug thereof, bezafibrate and optionally one or more pharmaceutically acceptable carriers.

In some embodiments, the methods of the present disclosure comprise administering to a subject in need thereof an effective amount of OCA, in combination with, and optionally one or more pharmaceutically acceptable carriers. In a further embodiment, the method comprises administering to a subject in need thereof an effective amount of OCA or a pharmaceutically acceptable salt, solvate, or amino acid, sulfate or glucuronide conjugate, or prodrug thereof in combination with BZF and optionally one or more pharmaceutically acceptable carriers.

In some embodiments, the subject is a mammal. In some embodiments, the mammal is a human

In one of the embodiments, OCA and BZF are administered in a two-way combination, i.e., without any therapeutic agent other than FXR agonist of the present disclosure and the fibrate of the present disclosure.

In the methods of the present disclosure the active substances (e.g., OCA and/or BZF) may be administered in single daily doses, or in two, three, four or more identical or different divided doses per day, and they may be administered simultaneously or at different times during the day.

In some embodiments, OCA and BZF are administered concurrently. In some embodiments, OCA and BZF are administered sequentially. For example, OCA is administered prior or subsequent to BZF. In some embodiments, OCA and BZF are administered as a fixed dose combination (FDC).

In some embodiments, a combination of OCA and BZF is administered orally, parenterally, or topically. In some embodiments, the combination is administered orally.

In some embodiments, the combination of OCA and BZF is provided in a single pharmaceutical composition with a pharmaceutical acceptable carrier (e.g., tablet, capsule, etc.).

In some embodiments, a combination comprises BZF in an amount of about 100 mg and OCA in an amount of between about 5 and about 10 mg. In one embodiment, OCA is in an amount of about 5 mg and BZF in an amount of about 100 mg. In another embodiment, OCA is in an amount of about 10 mg and BZF in an amount of about 100 mg.

In some embodiments, the combination of OCA and BZF is a fixed dose combination (FDC). In some embodiments, the FDC comprises bezafibrate in an amount of about 100 mg and OCA in an amount of between about 5 and about 10 mg. In one embodiment, the FDC comprises OCA is in an amount of about 5 mg and BZF in an amount of about 100 mg. In another embodiment, the FDC comprises OCA is in an amount of about 10 mg and BZF in an amount of 100 mg.

In some embodiments, a fixed dose combination (FDC) comprises from about 0.5 mg to about 30 mg of OCA. In some embodiments, the FDC comprises about 0.5 mg, 1 mg, about 2 mg, about 3 mg, about 5 mg, about 10 mg, about 15 mg, about 20 mg, about 25 mg, or about 30 mg of OCA. In some embodiments, the FDC comprises between 1 and 5 mg OCA. In some embodiments, the FDC comprises between 5 and 10 mg OCA. In some embodiments, the FDC comprises between 10 and 15 mg of OCA. In some embodiments the FDC comprises about 2.5 mg OCA. In some embodiments, the FDC comprises about 5 mg of OCA. In some embodiments, the tablet comprises about 10 mg of OCA. In some embodiments, the FDC comprises about 25 mg OCA.

In some embodiments, a fixed dose combination (FDC) comprises from about 50 mg to about 400 mg of bezafibrate (BZF). In some embodiments, the FDC comprises about 50 mg, about 100 mg, about 200 mg, about 300 mg, or about 400 mg of BZF. In some embodiments, the FDC comprises between about 50 mg and about 150 mg of BZF. In some embodiments, the FDC comprises between about 100 mg and about 200 mg of BZF. In some embodiments, the FDC comprises between about 100 mg and about 150 mg of BZF. In some embodiments, the FDC comprises between about 150 mg and about 200 mg of BZF. In some embodiments, the tablet comprises about 50 mg BZF. In some embodiments, the FDC comprises about 100 mg BZF. In some embodiments, the FDC comprises about 150 mg of BZF. In some embodiments, the FDC comprises about 200 mg of BZF. In some embodiments, the FDC comprises about 250 mg of BZF.

In some embodiments, the combination comprises OCA and BZF formulated for either immediate or modified (e.g., sustained) release. In some embodiments, the combination comprises OCA formulated for immediate release and BZF formulated for sustained release. In some embodiments, the combination comprises OCA formulated for immediate release and BZF formulated for immediate release. In some embodiments, the combination comprises OCA formulated for modified (e.g., sustained) release and BZF formulated for sustained release. In some embodiments, the combination comprises OCA formulated for modified (e.g., sustained) release and BZF formulated for immediate release.

In one aspect, the combination of OCA and BZF is administered for the treatment or prevention of a disease or condition, in place of UDCA to a subject who has an inadequate therapeutic response to UDCA (used alone or in combination with another active). In one aspect the disease or condition is PBC.

Pharmaceutical compositions of the present disclosure may be in any convenient form for oral administration, such as a tablet, capsule, powder, lozenge, pill, troche, elixir, lyophilized powder, solution, granule, suspension, emulsion, syrup or tincture. Slow-release, modified release, or delayed-release forms may also be prepared, for example in the form of coated particles, multi-layer tablets, capsules within capsules, tablets within capsules, or microgranules.

Solid forms for oral administration may contain pharmaceutically acceptable binders, sweeteners, disintegrating agents, diluents, flavoring agents, coating agents, preservatives, lubricants and/or time delay agents. Suitable binders include gum acacia, gelatin, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavoring agents include peppermint oil, oil of wintergreen, cherry, orange or raspberry flavoring. Suitable coating agents include polymers or copolymers or acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulfite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further include dispersing agents and/or suspending agents. Suitable suspending agents include sodium carboxymethylcellulose, methylcellulose, hydroxypropyl methylcellulose, polyvinylpyrrolidone, sodium alginate or cetyl alcohol. Suitable dispersing agents include lecithin, polyoxyethylene esters of fatty acids such as stearic acid, polyoxyethylene sorbitol mono-or di-oleate,-stearate or-laurate, polyoxyethylene sorbitan mono-or di-oleate,-stearate or-laurate and the like.

Emulsions for oral administration may further include one or more emulsifying agents. Suitable emulsifying agents include dispersing agents as exemplified above or natural gums such as gum acacia or gum tragacanth.

In some embodiments, the fibrate of the present disclosure is provided either in an immediate release tablet or in a sustained release tablet. In one of the embodiments, the fibrate of the present disclosure is provided in a sustained release tablet. In one of the embodiments, it is preferable for prolonged action that the tablet is in a sustained release format.

In some embodiments, a pharmaceutical composition of the disclosure is a dosage form which comprises a FXR agonist of the present disclosure or a pharmaceutically acceptable salt, solvate, or amino acid, sulfate or glucuronide conjugate, or prodrug thereof in a daily total amount of from 5-10 mg. In some embodiments, the total amount is orally administered once a day.

In some embodiments, a pharmaceutical composition of the disclosure is a dosage form which comprises a fibrate of the present disclosure in a daily total amount about 100 mg. In some embodiments, the total amount is orally administered once a day.

In some embodiments, a pharmaceutical compositions of the present disclosure can be used lifelong by the patient, prolonging survival and delaying liver transplantation. The reduction of hyperlipidemia and liver enzymes ensures reduction in the development of associated vascular disease. Because of the simplified dosing, the combined therapy of the present disclosure can be used in adjusting (increasing or decreasing) doses, depending on a patient's weight and clinical response. In one aspect, the combined therapy provides reduced side effect profile.

FXR agonists disclosed herein can be prepared by conventional methods (e.g., as described in U.S. Publication No. 2009/0062526; U.S. Pat. No. 7,138,390; WO 2006/122977; WO 2013/192097; U.S. Pat. No. 7,932,244; WO 2014/066819; WO 2014/184271; and WO 2017/062763).

The aspects of the present disclosure are further described with reference to the following numbered embodiments:

1. A method of treating or preventing an FXR mediated disease or condition in a subject in need thereof, comprising administering to the subject obeticholic acid (OCA) or a pharmaceutically acceptable salt, solvate, or amino acid, sulfate or glucuronide conjugate, or prodrug thereof in an amount of between about 5 and about 10 mg, and bezafibrate or a pharmaceutically acceptable salt or ester thereof in an amount between about 100 mg and about 400 mg.
2. The method of embodiment 1, wherein the obeticholic acid (OCA) or pharmaceutically acceptable salt, solvate, or amino acid, sulfate or glucuronide conjugate, or prodrug thereof is administered in an amount of about 5 mg.
3. The method of embodiment 1, wherein the obeticholic acid (OCA) or pharmaceutically acceptable salt, solvate, or amino acid, sulfate or glucuronide conjugate, or prodrug thereof is administered in an amount of about 10 mg.
4. The method of any one of embodiments 1-3, wherein the bezafibrate or pharmaceutically acceptable salt or ester thereof is administered in an amount of about 100 mg.
5. The method of any one of embodiments 1-3, wherein the bezafibrate or pharmaceutically acceptable salt or ester thereof is administered in an amount of about 400 mg.
6. The method of any one of embodiments 1-5, comprising administering to the subject an effective amount of OCA.
7. The method of any one of embodiments 1-6, comprising administering to the subject an effective amount of bezafibrate.
8. A method of treating or preventing an FXR mediated disease or condition in a subject in need thereof, comprising administering to the subject a first pharmaceutical composition comprising obeticholic acid (OCA) or a pharmaceutically acceptable salt, solvate, or amino acid, sulfate or glucuronide conjugate, or prodrug thereof in an amount of between about 5 and about 10 mg, and a second pharmaceutical composition comprising bezafibrate or a pharmaceutically acceptable salt or ester thereof in an amount between about 100 mg and about 400 mg.
9. The method of embodiment 8, wherein the first pharmaceutical composition comprises OCA.
10. The method of embodiment 8 or embodiment 9, wherein the second pharmaceutical composition comprises bezafibrate.
11. The method of any one of embodiments 8-10, wherein the first pharmaceutical composition comprises OCA or pharmaceutically acceptable salt, solvate, or amino acid, sulfate or glucuronide conjugate, or prodrug thereof in an amount of about 5 mg.
12. The method of any one of embodiments 8-10, wherein the first pharmaceutical composition comprises OCA or pharmaceutically acceptable salt, solvate, or amino acid, sulfate or glucuronide conjugate, or prodrug thereof in an amount of about 10 mg.
13. The method of any one of embodiments 8-12, wherein the second pharmaceutical composition comprises bezafibrate or pharmaceutically acceptable salt or ester thereof in an amount of about 100 mg.
14. The method of any one of embodiments 8-12, wherein the second pharmaceutical composition comprises bezafibrate or pharmaceutically acceptable salt or ester thereof in an amount of about 400 mg.
15. The method of any one of embodiments 8-14, wherein the first pharmaceutical composition is a tablet.
16. The method of any one of embodiments 8-15, wherein the second pharmaceutical composition is a tablet.
17. The method of any one of embodiments 8-16, wherein the first pharmaceutical composition is an immediate release form.
18. The method of any one of embodiments 8-16, wherein the first pharmaceutical composition is a sustained release tablet.
19. The method of any one of embodiments 8-18, wherein the second pharmaceutical composition is an immediate release form.
20. The method of any one of embodiments 8-18, wherein the second pharmaceutical composition is a sustained release tablet.
21. The method of any one of embodiments 1-20, wherein the FXR mediated disease or condition is selected from primary biliary cholangitis (PBC), primary sclerosing cholangitis (PSC), portal hypertension, bile acid diarrhea, a chronic liver disease, nonalcoholic fatty liver disease (NAFLD), nonalcoholic steatohepatitis (NASH), hepatitis C infection, an alcoholic liver disease, liver damage due to progressive fibrosis, liver fibrosis, drug-induced cholestasis, hereditary cholestasis, biliary atresia, and intrahepatic cholestasis of pregnancy.
22. The method of embodiment 21, wherein the FXR mediated disease or condition is chronic liver disease.
23. The method of embodiment 21, wherein the FXR mediated disease or condition is a cholestatic liver disease.
24. The method of embodiment 21, wherein the FXR mediated disease or condition is PBC.
25. The method of embodiment 21, wherein the FXR mediated disease or condition is NASH.
26. The method of embodiment 21, wherein the FXR mediated disease or condition is liver fibrosis.
27. The method of embodiment 21, wherein the FXR mediated disease or condition is liver fibrosis associated with NASH.
28. The method of any one of embodiments 1-27, wherein the OCA or pharmaceutically acceptable salt, solvate, or amino acid, sulfate or glucuronide conjugate, or prodrug thereof and the bezafibrate or pharmaceutically acceptable salt or ester thereof are administered sequentially.
29. The method of any one of embodiments 1-27, wherein the OCA or pharmaceutically acceptable salt, solvate, or amino acid, sulfate or glucuronide conjugate, or prodrug thereof and the bezafibrate or pharmaceutically acceptable salt or ester thereof are administered concurrently.
30. The method of any one of embodiments 1-28, wherein the OCA or pharmaceutically acceptable salt, solvate, or amino acid, sulfate or glucuronide conjugate, or prodrug thereof is administered prior to the administration of the bezafibrate or pharmaceutically acceptable salt or ester thereof.
31. The method of any one of embodiments 1-28, wherein the OCA or pharmaceutically acceptable salt, solvate, or amino acid, sulfate or glucuronide conjugate, or prodrug thereof is administered subsequent to the administration of the bezafibrate or pharmaceutically acceptable salt or ester thereof.
32. OCA for use in a method according to any one of embodiments 1-31.
33. A pharmaceutical composition comprising OCA and a pharmaceutically acceptable carrier or excipient for use in a method according to any one of embodiments 1-31.
34. OCA for use in the manufacture of a medicament for treating or preventing an FXR mediated disease or condition, according to the method of any one of embodiments 1-31.
35. Use of OCA for the treatment of an FXR mediated disease or condition according to the method of any one of embodiments 1-31.
36. Use of OCA in in the manufacture of a medicament for treating or preventing an FXR mediated disease or condition, according to the method of any one of embodiments 1-31.

### Definitions

For convenience, certain terms used in the specification, examples and appended claims are collected here.

The term “fibrate of the present disclosure” refers to bezafibrate and pharmaceutically acceptable salts or esters thereof.

Bezafibrate (BZF), a pan-peroxisome proliferator-activated receptor (PPAR) [α, δ, γ] agonist, was originally developed for treatment of hyperlipidemia and used for the prevention of cardiovascular disease. BZF also decreases serum hepatobiliary enzyme activity in individuals with and without cardiovascular disease and thus has been identified as a potential anticholestatic agent for the treatment of PBC with an inadequate response to UDCA.

OCA is a selective FXR agonist that has been shown to effect significant reductions in ALP in patients with PBC who demonstrated no or partial response to UDCA. As such, OCA has been conditionally approved for patients with PBC in combination with UDCA for those with an inadequate response to UDCA or who are intolerant to UDCA.

Without being bound to any theory, this disclosure relates to concomitant use of OCA and BZF which results in improved efficacy and tolerability compared to the previous PBC therapies and treatment with OCA alone.

The term “FXR agonist of the present disclosure” refers to OCA and pharmaceutically acceptable salts, solvates, or amino acid, sulfate or glucuronide conjugates, and prodrugs thereof.

As used herein, the term “obeticholic acid” or “OCA” refers to a compound having the chemical structure:

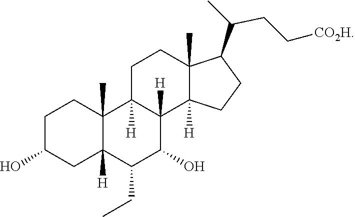

Obeticholic acid (OCA), a farnesoid X receptor (FXR) agonist and modified bile acid derived from the primary human bile acid chenodeoxycholic acid (CDCA), was developed for the treatment of PBC and to provide patients who have an inadequate response to or poor tolerance of UDCA, a novel treatment option that was safe and effective (Pellicciari R, Fiorucci S, Camaioni E, et al. 6alpha-ethyl-chenodeoxycholic acid (6 ECDCA), a potent and selective FXR agonist endowed with anticholestatic activity. J Med Chem. 2002;45:3569-3572).

Obeticholic acid is also referred to as 3α,7α-dihydroxy-6α-ethyl-5β-cholan-24-oic acid, 6α-ethyl-chenodeoxycholic acid, 6-ethyl-CDCA, 6ECDCA, cholan-24-oic acid, 6-ethyl-3,7-dihydroxy-(3α,5β,6α,7α), and can be prepared by the methods described in U.S. Publication No. 2009/0062526 A1, U.S. Pat. No. 7,138,390, and WO2006/122977. The CAS registry number for obeticholic acid is 459789-99-2.

The term “the compound” means OCA, or a pharmaceutically acceptable salt, solvate, or amino acid, sulfate or glucuronide conjugate, or prodrug thereof. Whenever the term is used in the context of the present disclosure it is to be understood that the reference is being made to a free form, an isotopically-labeled compound, a crystalline compound, a non-crystalline compound or a corresponding pharmaceutically acceptable salt or amino acid conjugates thereof, provided that such is possible and/or appropriate under the circumstances.

As used herein, the term “amino acid conjugates” refers to conjugates of OCA with any suitable amino acid. For example, such a suitable amino acid conjugate of OCA has the added advantage of enhanced integrity in bile or intestinal fluids. Suitable amino acids include but are not limited to glycine, taurine and sarcosine. Thus, the present disclosure encompasses the glycine, taurine and sarcosine conjugates of OCA.

“Treating” includes any effect, e.g., lessening, reducing, modulating, or eliminating, that results in the improvement of the condition, disease, disorder, etc. “Treating” or “treatment” of a disease state includes inhibiting the existing disease state, i.e., arresting the development of the disease state or its clinical symptoms, or relieving the disease state, i.e., causing temporary or permanent regression of the disease state or its clinical symptoms.

“Preventing” a disease state includes causing the clinical symptoms of the disease state not to develop in a subject that may be exposed to or predisposed to the disease state but does not yet experience or display symptoms of the disease state.

The term “inhibiting” or “inhibition” as used herein refers to any detectable positive effect on the progression of a disease or condition. Such a positive effect may include the delay in progression of at least one symptom or sign of the disease or condition, alleviation or reversal of the symptom(s) or sign(s) and slowing of the further worsening of the symptom(s) or sign(s).

“Disease state” means any disease, disorder, condition, symptom, or indication.

The term “effective amount” or “therapeutically effective amount” as used herein refers to an amount of an FXR agonist of the present disclosure or a fibrate of the present disclosure that produces an acute or chronic therapeutic effect upon appropriate dose administration, alone or in combination. In some embodiments, an effective amount or therapeutically effective amount of an FXR-activating ligand produces an acute or chronic therapeutic effect upon appropriate dose administration in combination with a fibrate of the present disclosure. The effect includes the prevention, correction, inhibition, or reversal of the symptoms, signs and underlying pathology of a disease/condition (e.g., fibrosis of the liver, kidney, or intestine) and related complications to any detectable extent. An “effective amount” or “therapeutically effective amount” varies depending on the FXR agonist of the present disclosure, the fibrate of the present disclosure, the disease and its severity, and the age, weight, etc., of the subject to be treated.

“Pharmacological effect” as used herein encompasses effects produced in the subject that achieve the intended purpose of a therapy. In some embodiments, a pharmacological effect means that primary indications of the subject being treated are prevented, alleviated, or reduced. For example, a pharmacological effect would be one that results in the prevention, alleviation or reduction of primary indications in a treated subject. In another embodiment, a pharmacological effect means that disorders or symptoms of the primary indications of the subject being treated are prevented, alleviated, or reduced. For example, a pharmacological effect would be one that results in the prevention, alleviation or reduction of the disorders or symptoms in a treated subject.

It is to be understood that the isomers arising from asymmetric carbon atoms (e.g., all enantiomers and diastereomers) are included within the scope of the disclosure, unless indicated otherwise. Such isomers can be obtained in substantially pure form by classical separation techniques and by stereochemically controlled synthesis.

A “pharmaceutical composition” is a formulation containing therapeutic agents such as an FXR agonist of the present disclosure and/or a fibrate of the present disclosure, in a form suitable for administration to a subject. In some embodiments, a pharmaceutical composition is in bulk or in unit dosage form. It can be advantageous to formulate compositions in dosage unit form for ease of administration and uniformity of dosage. Dosage unit form as used herein refers to physically discrete units suited as unitary dosages for the subject to be treated; each unit containing a predetermined quantity of active reagent calculated to produce the desired therapeutic effect in association with the required pharmaceutical carrier. The specification for the dosage unit forms of the disclosure is dictated by and directly dependent on the unique characteristics of the active agents and the particular therapeutic effect to be achieved, and the limitations in the art of compounding such an active agent for the treatment of individuals.

The term “unit dosage form” refers to physically discrete units suitable as unitary dosages for humans and other mammals, each unit containing a predetermined quantity of active material calculated to produce the desired therapeutic effect, in association with a suitable pharmaceutical excipient as described herein.

The unit dosage form is any of a variety of forms, including, for example, a capsule, an IV bag, a tablet, a single pump on an aerosol inhaler, or a vial. The quantity OCA or a pharmaceutically acceptable salt, solvate, or amino acid, sulfate or glucuronide conjugate, or prodrug thereof in a unit dose of composition is an effective amount and is varied according to the particular treatment involved and/or the fibrate used for the treatment. One skilled in the art will appreciate that it is sometimes necessary to make routine variations to the dosage depending on the age and condition of the patient. The dosage also depends on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for topical or transdermal administration of a compound of this disclosure include powders, sprays, ointments, pastes, creams, lotions, gels, solutions, patches and inhalants. In some embodiments, the FXR agonist of the present disclosure and/or fibrate of the present disclosure is mixed under sterile conditions with a pharmaceutically acceptable carrier, and with any preservatives, buffers, or propellants that are required.

The term “flash dose” refers to formulations that are rapidly dispersing dosage forms.

The term “immediate release” is defined as a release of a therapeutic agent (such as an FXR agonist of the present disclosure or a fibrate of the present disclosure) from a dosage form in a relatively brief period of time, generally up to about 60 minutes. The term “modified release” is defined to include delayed release, sustained release or extended release, and pulsed release. The term “pulsed release” is defined as a series of releases of drug from a dosage form. The term “sustained release” or “extended release” is defined as continuous release of a therapeutic agent from a dosage form over a prolonged period.

A “subject” includes mammals, e.g., humans, companion animals (e.g., dogs, cats, birds, and the like), farm animals (e.g., cows, sheep, pigs, horses, fowl, and the like), and laboratory animals (e.g., rats, mice, guinea pigs, birds, and the like). In some embodiments, the subject is a human. In one aspect, the subject is female. In one aspect, the subject is male.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier or excipient” means a carrier or excipient that is useful in preparing a pharmaceutical composition that is generally safe, non-toxic and neither biologically nor otherwise undesirable, and includes any excipient that is acceptable for veterinary use and/or human pharmaceutical use. A “pharmaceutically acceptable excipient” as used herein includes both one and more than one such excipient.

“Fibrosis” refers to a condition involving the development of excessive fibrous connective tissue, e.g., scar tissue, in a tissue or organ. Such generation of scar tissue may occur in response to infection, inflammation, or injury of the organ due to a disease, trauma, chemical toxicity, and so on. Fibrosis may develop in a variety of different tissues and organs, including the liver, kidney, intestine, lung, heart, etc.

As used herein, a “cholestatic condition” refers to any disease or condition in which bile excretion from the liver is impaired or blocked, which can occur either in the liver or in the bile ducts. Intrahepatic cholestasis (which occurs inside the liver) and extrahepatic cholestasis (which occurs outside the liver) are the two types of cholestatic conditions.

Clinical symptoms and signs of a cholestatic condition include itching (pruritus), fatigue, jaundiced skin or eyes, inability to digest certain foods, nausea, vomiting, pale stools, dark urine, and right upper quadrant abdominal pain. A patient with a cholestatic condition can be diagnosed and followed clinically based on a set of standard clinical laboratory tests, including measurement of levels of alkaline phosphatase, γ-glutamyl transpeptidase (GGT), 5′ nucleotidase, bilirubin, bile acids, and cholesterol in a patient's blood serum. Generally, a patient is diagnosed as having a cholestatic condition if serum levels of all three of the diagnostic markers: alkaline phosphatase, GGT, and 5′ nucleotidase, are considered abnormally elevated. The normal serum level of these markers may vary to some degree from laboratory to laboratory and from procedure to procedure, depending on the testing protocol. Thus, a physician is able to determine, based on the specific laboratory and test procedure, what an abnormally elevated blood level is for each of the markers. For example, a patient suffering from a cholestatic condition generally has greater than about 125 IU/L alkaline phosphatase, greater than about 65 IU/L GGT, and greater than about 17 NIL 5′ nucleotidase in the blood. Because of the variability in the level of serum markers, a cholestatic condition may be diagnosed on the basis of abnormal levels of these three markers in addition to at least one of the symptoms mentioned above, such as itching (pruritus).

Pruritus is an adverse event (AE) and must be graded for severity (i.e., intensity). Because pruritus is a subjective symptom and its occurrence and magnitude are not readily measured by objective tools, clinical judgment is applied to determine its severity and management in each subject. In order to assess the potential improvement in pruritus with treatment, baseline pruritus presence (yes/no) and severity is determined. Severity of Pruritus: 1=Mild (Mild or localized; topical intervention indicated); 2=Moderate (Intense or widespread; intermittent; skin changes from scratching (e.g., edema, papulation, excoriations, lichenification, oozing/crusts); oral intervention indicated; limiting instrumental activities of daily living); 3=Severe (Intense or widespread; constant; limiting self-care activities of daily living or sleep; oral corticosteroid or immunosuppressive therapy indicated). The present disclosure also relates to a method of reducing adverse events, such as pruritus, comprising administering the disclosed combination. The present disclosure also relates to a method of mitigating adverse events elicited or caused by OCA monotherapy, such as pruritus, comprising administering the disclosed combination of OCA and BZF.

The term “primary biliary cholangitis”, previously called “primary biliary cirrhosis”, often abbreviated PBC, is an autoimmune disease of the liver marked by the slow progressive destruction of the small bile ducts of the liver, with the intralobular ducts (Canals of Hering) affected early in the disease. When these ducts are damaged, bile builds up in the liver (cholestasis) and over time damages the tissue. This can lead to scarring, fibrosis and cirrhosis. PBC is characterized by interlobular bile duct destruction. Histopathologic findings of primary biliary cirrhosis include: inflammation of the bile ducts, characterized by intraepithelial lymphocytes, and periductal epithelioid granulomata. There are 4 stages of PBC.
Stage 1—Portal Stage: Normal sized triads; portal inflammation, subtle bile duct damage. Granulomas are often detected in this stage.
Stage 2—Periportal Stage: Enlarged triads; periportal fibrosis and/or inflammation. Typically, this stage is characterized by the finding of a proliferation of small bile ducts.
Stage 3—Septal Stage: Active and/or passive fibrous septa.
Stage 4—Biliary Cirrhosis: Nodules present; garland

The term “primary sclerosing cholangitis” (PSC) is a disease of the bile ducts that causes inflammation and subsequent obstruction of bile ducts both at an intrahepatic (inside the liver) and extrahepatic (outside the liver) level. The inflammation impedes the flow of bile to the gut, which can ultimately lead to cirrhosis of the liver, liver failure and liver cancer.

The term “organ” refers to a differentiated structure (as in a heart, lung, kidney, liver, etc.) consisting of cells and tissues and performing some specific function in an organism. This term also encompasses bodily parts performing a function or cooperating in an activity (e.g., an eye and related structures that make up the visual organs). The term “organ” further encompasses any partial structure of differentiated cells and tissues that is potentially capable of developing into a complete structure (e.g., a lobe or a section of a liver).

All publications and patent documents cited herein are hereby incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the description and examples provided herein are for purposes of illustration and not limitation of the claims that follow.

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the present specification controls. All percentages and ratios used herein, unless otherwise indicated, are by weight.

### EXAMPLES

Example 1: Clinical Trial to Determine the Effects of OCA in Combination with BZF on PBC

### Study Design

A Phase 2a, randomized, double blind (DB), active-controlled, parallel-group study is being conducted to evaluate the efficacy, safety, and tolerability of OCA (5 mg/day) administered in combination with two different BZF doses (100 and 400 mg/day) compared to BZF alone (100 and 400 mg/day) in up to 60 subjects with PBC over at least 12 weeks. Subjects are randomized (1:1:1:1) to the following 4 treatments: Treatment A: BZF 100 mg IR; Treatment B: BZF 400 mg IR; Treatment C: OCA 5 mg+BZF 100 mg IR; and Treatment D: OCA 5 mg+BZF 400 mg IR. This study uses 100-mg and 200-mg immediate release (IR) formulation tablets of BZF.

The primary objective is to assess the effects of the combination of OCA and BZF on ALP in comparison to BZF alone in subjects with PBC.

The secondary objectives are to assess the effects of the combination of OCA plus BZF versus BZF alone in subjects with PBC on the following: (a) response and normalization rates of biochemical disease markers, including GGT, ALT, AST, and total and conjugated bilirubin; (b) biomarkers of bile acid synthesis and homeostasis, including 7α-hydroxy-4-cholesten-3-one (C4) and bile acids; and (c) safety and tolerability

The additional objectives are to assess the combination of OCA plus BZF versus BZF alone in subjects with PBC on the following: (a) pharmacokinetics (PK); (b) PK/pharmacodynamic (PD) relationships with pharmacogenomics (PGx) biomarkers and safety/efficacy markers; (c) disease-specific symptoms as assessed by health-related quality of life questionnaires [PBC-40, pruritus visual analog scale (VAS), numerical rating scale (NRS), QOL and Fatigue Impact Scale (FIS)]; (d) noninvasive assessments of liver fibrosis, including transient elastography (TE) and enhanced liver fibrosis (ELF) score; markers of collagen formation and degradation, including type III pro-collagen (Pro-C3), type V pro-collagen (Pro-C5), type III collagen (C3M), and type IV collagen (C4M); as well as Fibrosis-4 (Fib4) and AST to platelet ratio index (APRI); (e) DB Phase: Percentage of participants at Week 12 with ALP<1.67× upper limit of normal (ULN) and total bilirubin≤ULN and ALP decrease of ≥15% from baseline; (f) LTSE Phase: Percentage of participants at Weeks 24, 36, 48 with ALP <1.67× ULN and total bilirubin ≤ULN and ALP decrease of ≥15% from baseline (DB Week 12 is the baseline for the LTSE Phase); (g) noninvasive assessment of liver function (HepQuant SHUNT) (for qualified US sites only); and (h) disease severity scores (GLOBE, UK-PBC scores, and model of end-stage liver disease [MELD]).

### Inclusion and Exclusion Criteria

### Diagnosis and Main Criteria for Inclusion

1. A definite or probable diagnosis of PBC (consistent with the EASL and the AASLD guidelines [Lindor 2009a; EASL 2017]), as demonstrated by the presence of at least 2 of the following 3 diagnostic factors:
History of elevated ALP levels for at least 6 months
Positive antimitochondrial antibody (AMA) titer, or if AMA negative or low titer (≤1:80), PBC-specific antibodies (anti-GP210 and/or anti-SP100) and/or antibodies against the major M2 components (pyruvate dehydrogenase-E2, 2-oxo-glutaric acid dehydrogenase complex)
Liver biopsy results consistent with PBC (collected at any time before Screening. At least one of the following qualifying biochemistry values (the mean of both screening visits):
ALP≥1.67× upper limit of normal (ULN) (including up to 10% of subjects with ALP>5× ULN)
ALP≥1.5× ULN but ≤1.67× ULN (will not exceed 25% of subjects enrolled in the study).
Total bilirubin>ULN but <2× ULN
   2. At least one of the following qualifying biochemistry values (the mean of both Screening visits):
ALP>1.67× ULN (including up to 10% of subjects with ALP>5× ULN).
ALP≥1.5× ULN but ≤1.67× ULN (will not exceed 25% of subjects enrolled in the study)
Total bilirubin>ULN but <2× ULN
   3. Age≥18 years
   4. Taking UDCA for at least 12 months (stable dose for ≥3 months) before Day 1 or no UDCA for 3 months before Day 1.
   5. Contraception: Female subjects must be postmenopausal, surgically sterile, or, if premenopausal (and not surgically sterile), be prepared to use ≥1 highly effective method of contraception during the study and for 30 days after the end of treatment. Highly effective methods of contraception per the Clinical Trials Facilitation and Coordination Group (CTFG) guidelines are those that alone or in combination results in a failure rate of less than 1% per year when used consistently and correctly. Highly effective methods of contraception are as follows:
Intrauterine device
   intrauterine device (IUD)
   intrauterine hormone-releasing system (IUS)
Bilateral tubal occlusion
Vasectomy (partner)
Combined (estrogen and progestogen containing) hormonal contraception (e.g., oral, intravaginal or transdermal) associated with inhibition of ovulation. If oral contraceptives are used, they must be used in combination with a male or female condom. Female subjects should be on the hormone contraception for at least 8 days prior to Day 1.
Progestogen-only hormonal contraception (e.g. oral, injectable or implantable) associated with inhibition of ovulation. If oral contraceptives are used, they must be used in combination with a male or female condom. Female subjects should be on the hormone contraception for at least 8 days prior to Day 1.
Sexual abstinence, if in line with the preferred and usual lifestyle of the subject (where abstinence is defined as refraining from heterosexual intercourse during the entire period of risk associated with study treatments).
   6. Must provide written informed consent and agree to comply with the study protocol.

### Key Exclusion Criteria

1. History or presence of other concomitant liver diseases including any of the following:
Hepatitis C virus (HCV) infection and ribonucleic acid positive
Active Hepatitis B virus (HBV) infection; however, subjects who have seroconverted [hepatitis B surface antigen negative (sAg−); hepatitis B surface antibody positive (sAB+); hepatitis B e-antigen negative (eAg−), hepatitis B e-antibody positive (eAB+; HBV DNA negative] may be included in this study
   Subjects should be excluded if positive HBV DNA orHCV RNA
Primary sclerosing cholangitis
Alcoholic liver disease
Definite autoimmune liver disease or PBC-AIH overlap
Non-Alcoholic Steatohepatitis (NASH)
Gilbert's Syndrome (due to interpretability of bilirubin levels)
Rare liver conditions such as Wilson's disease, hemochromatosis, alpha-one anti-trypsin deficiency, or other conditions
   2. Clinical complications of PBC including:
History of liver transplant, current placement on a liver transplant list, or current MELD score>12. Subjects who are placed on a transplant list despite a relatively early disease stage (for example per regional guidelines) may be eligible as long as they do not meet any of the other exclusion criteria
   3. History or presence of any of the following decompensating events:
   CP-Score ≥7
Clinical evidence of decompensated liver disease or obvious portal hypertension
   Varices
   Ascites/hepatic hydrothorax
   Spontaneous bacterial peritonitis
   Hepatic encephalopathy
   Jaundice (with total bilirubin>3 mg/dL [>51.3 μmol/L])
Biochemical evidence of hepatic impairment, decompensation, or injury including at least one of the following:
   Total bilirubin>3 mg/dL (>51.3 μmol/L)
   INR>1.7
   Albumin<3.5 g/dL
   MELD>12
   ALT>5× ULN.
Known or suspected hepatocellular carcinoma (HCC)
Prior trans-jugular intrahepatic portosystemic or peritoneovenous shunt procedure
Hepatorenal syndrome (type I or II) or serum creatinine >2 mg/dL [178 μmol/L] at Screening Visits 1 and 2
Portopulmonary hypertension
Hepatopulmonary syndrome
   4. Medical conditions that may cause non-hepatic increases in ALP (e.g., Paget's disease) or which may diminish life expectancy to <2 years, including known cancers (except carcinomas in situ or other stable, relatively benign conditions)
   5. Presence of any other disease or condition that interferes with the absorption, distribution, metabolism, or excretion of drugs including bile salt metabolism in the intestine (e.g., inflammatory bowel disease or gastric bypass procedure [gastric lap band is acceptable])
   6. History of or current gallbladder diseases:
Cholelithiasis or cholecystitis without prior cholecystectomy
Choledocholithiasis without documented complete stone removal (e.g. via ERCP) and cholecystectomy
Any size symptomatic gallbladder polyp
Gallbladder polyp >5 mm
   7. History or current pancreatitis
   8. History of drug-induced myopathy
   9. Chronic kidney disease (CKD) (serum creatinine >1.5 mg/100 mL (>135 μmol/L); creatinine clearance <60 mL/min) or undergoing dialysis
   10. Platelet count <150,000/mL at Screening Visits 1 and 2 (an average of both screening visits)
   11. Known history of human immunodeficiency virus (HIV) infection
   12. History or presence of clinically concerning cardiac arrhythmias likely to affect survival during the study, or Screening (pretreatment) QT or QTc interval of >450 milliseconds for males and >470 milliseconds for females
   13. Severe pruritus or required systemic treatment for pruritus (e.g., with bile acid sequestrants [BAS] or rifampicin) within 2 months of Day 1. Subjects who plan to use or will use BAS during the study will not be eligible.
   14. History of known or suspected clinically significant (CS) hypersensitivity to OCA, BZF, or other fibrates or any of their components
   15. Known photoallergic or phototoxic reactions to fibrates
   16. If female, known pregnancy, or has a positive urine pregnancy test (confirmed by a positive serum pregnancy test), or lactating
   17. Other CS medical conditions that are not well controlled or for which medication needs are anticipated to change during the study (e.g., Type 2 diabetes mellitus, hypothyroidism, nephritic syndrome, dysproteinemia, obstructive biliary disease, dyslipidemia) and/or up to the discretion of the Principal Investigator
   18. Treatment with the following medications 30 days before Day 1 or plans to use these medications during the study: azathioprine, colchicine, cyclosporine, methotrexate, mycophenolate mofetil, pentoxifylline, statins, budesonide and other systemic corticosteroids, monoamine oxidase inhibitors (MAOIs), and potentially hepatotoxic drugs (including α-methyl-dopa, sodium valproic acid, isoniazid, and nitrofurantoin)
   19. Treatment with the following medications 12 months before Day 1 or plans to use these medications during the study: antibodies or immunotherapy directed against interleukins or other cytokines or chemokines
   20. Participation in a study of another investigational medicine or device within 30 days before Screening
   21. Treatment with commercially available OCA or participation in a previous study involving OCA within 6 months before Screening
   22. Unable to tolerate BZF or other fibrates, treatment with commercially available fibrates or participation in a previous study involving fibrates within 3 months before Screening
   23. History of or ongoing alcohol or drug abuse within 1 year before Day 1. Subjects with moderate alcohol use within the past 3 months will be excluded from this study. Moderate alcohol consumption is defined as 1 standard drink per day for women and 2 drinks per day for men; whereby 1 standard drink is equivalent to: 12 oz beer (5% alcohol); 5 ounces of wine (12% alcohol), and 1.5 ounces of 80 proof (40% alcohol).
   24. History of noncompliance with medical regimens, or is considered not able to meet the requirements as specified in the protocol at the Screening visits and throughout the duration of the study
   25. Blood or plasma donation within 30 days before Day 1
   26. Mental instability or incompetence, such that the validity of informed consent or ability to be compliant with the study is uncertain
   27. A CK value at Screening ≥5× ULN or any abnormal laboratory value that is considered CS
   28. Known or suspected nephrotic syndrome based on the following diagnostic criteria (both Type 1 and Type 2):
Proteinuria, spot urine protein: albumin/creatinine ratio (ACR) of >300 mg/mmol (subjects with normal renal function and ACR values analyzed to be below the lower limits of detection are permitted)
Serum albumin <25 g/l
Clinical evidence of peripheral edema
Severe hyperlipidemia (total cholesterol above >10 mmol/l)
   The following exclusion criteria will only apply for subjects who are participating in the HepQuant SHUNT procedure:
   29. Subjects with a history of known or suspected hypersensitivity to any ingredient in human albumin preparations
   30. Subjects with uncontrolled hypertension (defined as a diastolic blood pressure of 110 mmHg or higher)
   31. Subjects with extensive resection of large segments of small intestine (short gut) or severe gastroparesis
   32. Subjects on either a non-selective beta blocker or an angiotensin-converting enzyme (ACE) inhibitor or angiotensin II receptor blocker (ARB) who are unwilling to delay taking their normal dose the morning of their testing

### Outcomes/Endpoints

The primary efficacy endpoint is the change in ALP from baseline to Week 12 in the DB Phase. The Primary Efficacy Analysis for the DB Phase is conducted using the mITT population when all subjects have completed the DB Phase. Analyses of change in ALP are carried out using a mixed-effect repeated measures model (MMRM) to evaluate the effect of treatment groups over time based on the mITT population. The dependent variable is the change in ALP from baseline. The model includes the terms of treatment group, visit, treatment by visit interaction, randomization stratification variables as factors and baseline value as covariate. The primary efficacy endpoint is also analyzed in the Per-Protocol Population.

### Screening Phase

Subjects are screened for a period of up to 8-weeks before being randomized into the study to allow for the collection of repeat serum chemistry samples (at least 2 weeks apart) for verification of inclusion/exclusion criteria and to establish baseline (including Day 1).

### Double-Blind Phase

Subjects who meet the entry requirements are randomized in a 1:1:1:1 ratio on Day 1 to receive either Treatment A (BZF 100 mg IR once daily; Treatment B (BZF 400 mg IR once daily); Treatment C (OCA 5 mg once daily+BZF 100 mg IR once daily); or Treatment D (OCA 5 mg once daily+BZF 400 mg IR once daily).

Subjects who are randomized to Treatment A or B receive 100 or 400 mg, respectively, of BZF alone and those randomized to Treatment C or D (combination groups) receive OCA 5 mg once daily and either 100 or 400 mg, respectively, of BZF. The DB Phase is from Day 1 through Week 12; eligible subjects will then continue into the LTSE Phase through the end of the study.

During PK assessments, a single blood draw is performed at predose on Day 1, and on study visits on Weeks 2, 6, 8, 10 and 12 (all single trough blood draws). At Week 4 study visit, a predose (trough PK) draw is taken and additional draws are taken at 0.25, 0.5, 0.75, 1, 1.5, 2, 2.5, 3, 4, 5 and 6 hours postdose for subjects with am dosing/am visit (from previous day), or at 18, 20, and 22 hours postdose on Week 4 for subjects with pm dosing (from previous day). Investigational products (IP) are taken orally once daily for the duration of the study. Subjects are instructed to begin dosing on Day 1 and to take IP at approximately the same time each day (morning dosing for the entire study is preferred to align with all study visits). Subjects are instructed to swallow all tablets; IP must not be chewed, divided, or crushed and must be taken with a sufficient amount of water. At study visits, all doses of IP (and of UDCA, for subjects on UDCA) are administered following a fast of at least 8 hours before dosing.

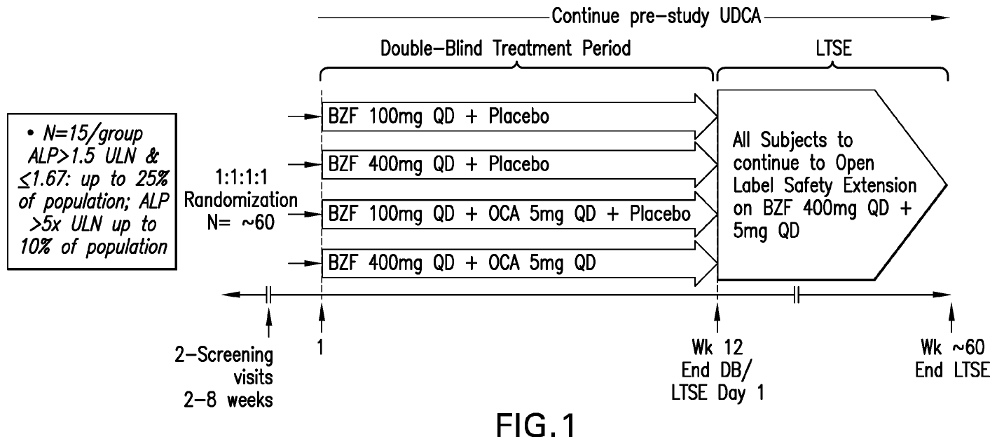

To preserve the study blind, appearance-matched placebo tablets for OCA and/or BZF are administered to subjects in each treatment group as shown in FIG. 1 during the treatment period. ALP=alkaline phosphatase; BZF=bezafibrate; DB=double blind; LTSE=long-term safety extension; N=number of subjects; OCA=obeticholic acid; QD=once daily; UDCA=ursodeoxycholic acid; ULN=upper limit of normal; Wk=week; Placebo=either OCA or BZF tablets. The screening phase is two weeks to a maximum of eight weeks. Subjects taking UDCA at the time of enrollment remain on their stable dose of UDCA during the study. Subjects who continue to meet protocol requirements transition to an LTSE at the completion of DB Phase. The dose for all subjects during the LTSE is 400 mg of BZF plus 5 mg OCA. LTSE Day 1 will be the last visit in the DB Phase (Weck 12).

Following completion of the 12-week DB Phase, subjects who continue to meet protocol requirements will transition to the LTSE Phase and receive the same dosing regimen of OCA 5 mg once daily+BZF 400 mg IR once daily (Treatment D in the DB Phase).

Randomization is stratified at baseline by the following:
Total bilirubin levels (≤0.7× or >0.7× ULN but <2× ULN).

This stratification is to ensure a balance of baseline disease severity between treatment arms (Lammers W J, van Buuren H R, Hirschfield G M, et al. Levels of alkaline phosphatase and bilirubin are surrogate end points of outcomes of patients with primary biliary cirrhosis: an international follow-up study. Gastroenterology. 2014;147(6):1338-1349)

Studies examining OCA in subjects with PBC demonstrate OCA's underlying efficacy occurring at ALP ≥1.67× ULN. The efficacy profile observed at this ALP level in the 12-month DB Phase 3 study in subjects treated with OCA remained durable over approximately 3 to 5 years of the long-term extension period (Trauner M, et. al. Long-term efficacy and safety of obeticholic acid for patients with primary biliary cholangitis: 3-year results of an international open-label extension study. Lancet Gastroenterol Hepatol. 2019Jun;4(6):445-453). Conversely, in subjects with PBC treated with BZF from the BEZURSO study over a 24-month period had a baseline ALP entry-criteria of ≥1.5× ULN (Corpechot C, Abenavoli L, Rabahi N, et al. Biochemical response to ursodeoxycholic acid and long-term prognosis in primary biliary cirrhosis. Hepatology. 2008;48:871-877).

Randomization is therefore also be stratified by ALP at baseline by the following:
ALP (≥1.5× ULN but ≤1.67× ULN or >1.67× ULN)
ALP>1.67× ULN (remaining subjects enrolled)

The percentage of subjects with baseline ALP≥1.5× ULN but ≤1.67× ULN does not exceed 25% and the percentage of subjects with baseline ALP>5× ULN does not exceed 10%.

After the Day 1 Visit, subsequent study visits during the DB Phase occurs at approximately Weeks 2, 4, 6, 8, 10 and 12 (end of DB Phase/Day 1 of LTSE) for the assessment of efficacy, safety, tolerability, and PK. These visits also allow for the assessment of any AEs, changes to concomitant medications and/or new medications that have been initiated, medical/surgical procedures, and to verify that the subject is dosing as directed. An evaluation of available efficacy and safety data may occur periodically during both the DB and LTSE Phases.

### LTSE Phase

Subjects who continue to meet protocol requirements will transition directly to the LTSE Phase upon completion of the Week 12 visit of the DB Phase. The end of the 12-week DB Phase becomes Day 1 of the LTSE Phase. All subjects will transition to the LTSE Phase on the OCA 5 mg once daily+BZF 400 mg once daily dose (Treatment D of the DB Phase) for the remainder of the study.

### Study Duration

The total duration of the study per subject is a minimum of approximately 62 weeks and a maximum of approximately 68 weeks, including a Screening Phase (a minimum of 2 weeks and up to 8 weeks), a DB Phase of 12 weeks, and an LTSE Phase of 48 weeks. Subjects who continue to meet protocol requirements will transition directly into the LTSE Phase once they complete the 12-week DB Phase.

### Number of Subjects

Up to 60 subjects (15 per group) are enrolled and randomized in a 1:1:1:1 ratio to 1 of 4 treatment arms (Treatments A, B, C, and D).

### Planned Dosing Regimen

Subjects are randomly assigned in a 1:1:1:1 ratio to receive one of the following treatments during the 12-week DB Phase:

| | |
|---|---|
| Treatment A: | OCA placebo tablet |
| BZF 100 mg IR tablet once daily | BZF 100 mg IR tablet |
| | BZF placebo tablet |
| Treatment B: | OCA placebo tablet |
| BZF 400 mg IR tablet once daily | BZF 200 mg IR tablet |
| | BZF 200 mg IR tablet |
| Treatment C: | OCA 5 mg tablet |
| OCA 5 mg once daily + BZF 100 | BZF 100 mg IR tablet |
| mg IR once daily | BZF placebo tablet |
| Treatment D: | OCA 5 mg tablet |
| OCA 5 mg once daily + BZF 400 | BZF 200 mg IR tablet |
| mg IR once daily | BZF 200 mg IR tablet |

Subjects who continue to meet protocol requirements will transition directly to the LTSE Phase upon completion of the Week 12 visit of the DB Phase. All subjects who transition to the LTSE Phase (e.g., will receive the OCA 5 mg once daily+BZF 400 mg once daily dose) (same as Treatment D of the DB Phase) for the remainder of the study.

### Monitoring and Management of Potential Hepatic Injury and or Disease Progression

Given the chronic and progressive nature of PBC, it is important to monitor for potential hepatic injury, disease progression, and/or hepatic decompensation. Child-Pugh and MELD scores are reviewed at each visit where labs are drawn. Child Pugh Scores are only applied in subjects who have evidence of cirrhosis at Screening or progression to cirrhosis during the study based on criteria presented below. In addition, AEs, signs and symptoms of potential hepatic injury or decompensation, and laboratory values are also reviewed at regular intervals. Based on the assessments of signs and symptoms of hepatic injury and liver biochemistry, the IP (OCA or BZF) may be interrupted or discontinued per the criteria discussed below.

### Signs and Symptoms of Hepatic Injury or Decompensation

Subjects are instructed to contact study personnel immediately if they notice any of the following signs and symptoms or have healthcare interactions outside of the study setting:
Specific signs and symptoms of liver impairment: e.g., yellowing of the skin or the whites of eyes, pale-colored stools, urine color change from pale to deep amber (dark) (reflecting impaired bilirubin metabolism)
More general signs and symptoms of ascites and encephalopathy: e.g., confusion, swelling of the legs or abdomen
Non-specific signs and symptoms of impaired health: e.g., nausea, vomiting,. abdominal pain, diarrhea, weight loss, fever, chills, worsening or new fatigue, weakness, loss of appetite
Significant intercurrent illnesses such as gastroenteritis may exacerbate hepatic injury or decompensation (e.g., established severe abdominal pain, vomiting, and persistent diarrhea) and should be an indication for prompt IP interruption and complete subject evaluation.

### Other Symptoms

Worsening of renal function (e.g. reduced urine output) or likely dehydration
New or worsening baseline myalgias or likely myositis

### Healthcare Provider (HCP) Interactions

Visits to primary HCP or referral to any specialist for any new symptoms (other than ongoing care for stable comorbidities)
New medications or changes to current medications prescribed from HCP or any new over the counter medications or herbal supplements
Laboratory procedures or assessments performed by an HCP

### Assessments for Suspected Hepatic and or Muscular Injury or Potential Hepatic Decompensation

Liver biochemistry and serum CPK is assessed to evaluate for potential hepatic and/or muscular injury or hepatic decompensation. These assessments are performed at:
Each protocol-specified visit
Unscheduled visits as needed in the event of signs or symptoms of suspected hepatic injury or decompensation or if laboratory alerts have been triggered

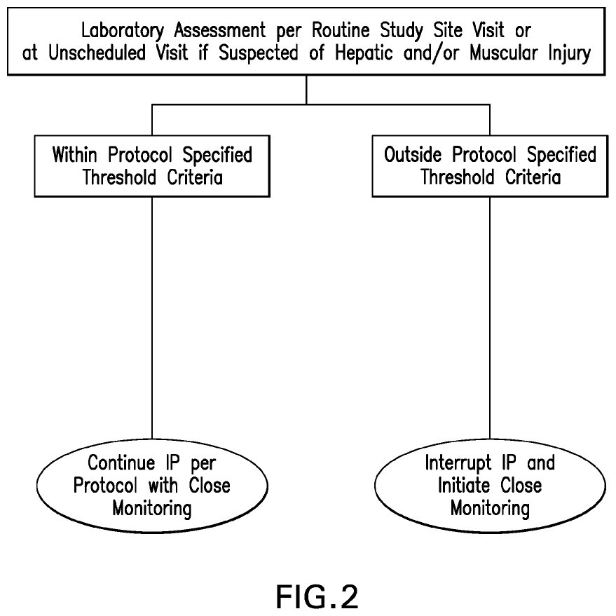

The process for assessing criteria is presented in FIG. 2. IP=investigational product. Laboratory assessments include ALT, ALP, AST, GGT, direct and total bilirubin, INR and CPK. PK sampling is conducted within 7 days of interruption; close monitoring including physical exams and repeat biochemistry labs occur as often as deemed appropriate. The specific threshold(s) for each laboratory analyte to trigger mandatory repeat testing and potentially a complete subject evaluation (depending on the repeat result) are summarized in Table 2.

TABLE 1
Laboratory Criteria for Monitoring of Suspected Hepatic and
Muscular Injury for Interruption^{a} of Investigational Product

| Baseline^{b} | Treatment Emergent | Liver Signs and Symptoms^{c} |
|---|---|---|
| Any value | Any hepatic enzyme elevation (TB, ALP, AST, | Yes |
| | ALT, GGT) | |
| ALT or AST Normal | ALT or AST ≥ 3x ULN and TB ≥ 2x BL | Y or N |
| | ALT or AST ≥ 3x ULN and INR > 1.5 | Y or N |
| | ALT or AST ≥ 5x ULN and Any TB | Y or N |
| ALT or AST Elevated | ALT or AST ≥ 2x BL and TB ≥ 2x BL | Y or N |
| | ALT or AST ≥ 2x BL and INR > 1.5 | Y or N |
| | ALT or AST ≥ 3x BL (or ≥300, whichever first) | Y or N |
| | and Any TB | |
| TB ≤ ULN | TB ≥ 2x BL and > ULN | Y or N |
| TB > ULN | TB ≥ 1.5x BL or ≥3.0 mg/dL, whichever occurs | Y or N |
| | first | |
| ALP ≤ ULN | ALP ≥ 2x BL and > ULN | Y or N |
| ALP > ULN | ALP ≥ 2x BL or ≥1000 U/L, whichever occurs | Y or N |
| | first | |
| CPK | >5x ULN or >3x ULN with symptoms of | N/A |
| | rhabdomyolysis^{d, e} | |

Abbreviations: ALP = alkaline phosphatase;
ALT = alanine aminotransferase;
AST = aspartate aminotransferase;
DB = direct bilirubin;
TB = total bilirubin;
ULN = upper limit of normal
^{a}Interrupt study drug; Initiate workup of competing etiologies and close monitoring (repeat labs and exam 2-3 times per week), including repeat labs initially. Study drug can be restarted after a minimum of 30 days if another etiology is identified, liver enzymes return to baseline, lab abnormalities are determined not to be due to DILI, no symptoms, and approved. If study drug is restarted after 30 days, repeat labs in 2-5 days for ALT, AST, and TB or 7-10 days for ALP as indicated and continue close monitoring.
^{b}New baseline values for DILI monitoring for ALT, AST, ALP, TB should be determined based upon average of month 3 and 6 values if average is >50% decrease from original baseline.
^{c}Liver signs and symptoms to include: new onset severe fatigue or significant worsening of baseline fatigue; new onset severe pruritus or significant worsening on baseline pruritus; nausea, vomiting, RUQ pain; fever, rash, or >5%
eosinophilia; new onset of hepatic decompensation (e.g. ascites, variceal bleeding, hepatic encephalopathy)
^{d}Symptoms of rhabdomyolysis may include muscle swelling; weak, tender, sore muscles; dark urine; fever; tachycardia; nausea and vomiting; confusion; dehydration; abdominal pain.
^{e}If IP interrupted due to CPK trigger, ensure follow CPK and creatinine/renal function in addition to liver biochemistries with close monitoring as above; same criteria apply to considering re-initiation of IP.

### Clinical Criteria for Monitoring for Potential Hepatic Decompensation Events

Subjects are monitored for potential hepatic decompensation events throughout the study. Investigational product is to be permanently discontinued with the development of any clinical events listed in Table 2.

Subjects who experience hepatic decompensation events are closely monitored until normalization or stabilization using good clinical judgement, and, per FDA guidance for observation of potential DILI, should include, but not be limited to, a thorough clinical evaluation including repeat liver enzymes and tests of liver function two to three times weekly with subsequent frequency to be determined by clinical status; complete history and physical; evaluation of concomitant drugs, alcohol, illicit drug use; concomitant liver conditions including viral hepatitis; environmental exposures; consider gastroenterology or hepatology consultation.

After stabilization, subjects continue with scheduled study visits for safety follow up. During the DB Phase, monitoring will occur on a bi-monthly basis from Day 1. Subjects who experience a decompensation event during the DB phase will not be eligible to enroll in the LTSE.

TABLE 2
Clinical Criteria for Monitoring Potential Hepatic Decompensation
Events and Mandatory Discontinuation of Investigational Product

Liver Transplant, under evaluation for liver transplant, or listed for transplant
Progression to CP score ≥7
New onset of any of the following:
Ascites
Hepatic hydrothorax
Hepatic encephalopathy - requires hospitalization and/or lactulose and/or rifaximin
Variceal bleeding
Jaundice (with total bilirubin >3 mg/dL [>51.3 μmol/L])
MELD ≥15
Hepatocellular carcinoma (HCC)
Any liver-related event requiring hospitalization and treatment
Hepatorenal syndrome Type 1 or Type 2, hepatopulmonary syndrome, or portopulmonary
syndrome
CP = Child-Pugh;
HCC = hepatocellular carcinoma;
MELD = Model for End-stage Liver Disease

### Child-Pugh Score

Child-Pugh (CP) Score (Pugh R N, Murray-Lyon I M, Dawson J L, et al. Transection of the oesophagus for bleeding oesophageal varices. Br J Surg. 1973 Aug;60(8):646-649, Lucey M R, Brown K A, Everson G T, et al. Minimal criteria for placement of adults on the liver transplant waiting list: a report of a national conference organized by the American Society of Transplant Physicians and the American Association for the Study of Liver Diseases. Liver Transpl Surg. 1997 Nov;3(6):628-637) is calculated and reported within the electronic data capture (EDC) system based on data entered into the eCRF by adding the scores from the five factors outlined in Table 4 and can range from 5 to 15. Although CP score calculation will automatically be computed in all subjects (at all times data is obtained for the eCRF), it should only be applied to subjects who meet the criteria for progression to cirrhosis. Dose adjustment or discontinuation is not considered based solely on the CP score in subjects who do not meet criteria for presence of cirrhosis.

A total score of 5 to 6 is considered Class A (mild, well-compensated disease); a score of 7 to 9 is Class B (moderate, significant functional compromise); and a score of 10 and above is Class C (severe, decompensated disease). The calculation of the CP Score includes assessments of ascites and hepatic encephalopathy, which may be assessed during the AE review at the scheduled visits, as well as total bilirubin, serum albumin, and prothrombin time (PT), the data for which will be obtained from the central laboratory.

TABLE 3
Child-Pugh Scoring System

| | | Points | | |
|---|---|---|---|---|
| Factor | Units | 1 | 2 | 3 |
| Serum bilirubin | μmol/L | <34 | 34-50 | >50 |
| | mg/dL | <2.0 | 2.0-3.0 | >3.0 |
| Serum albumin | g/L | >35 | 28-35 | <28 |
| | g/dL | >3.5 | 2.8-3.5 | <2.8 |
| Prothrombin time | Seconds | 0-3 | 4-6 | >6 |
| | prolonged | | | |
| | INR | <1.7 | 1.7-2.3 | >2.3 |
| Ascites | | None | Mild | Moderate-Severe |
| Hepatic | | No | Grade 1 or 2 | Grade 3 or 4 |
| encephalopathy^{a} | | | | |

CP = Child-Pugh;
INR = international normalized ratio
The CP score is calculated by adding the scores for the 5 factors and can range from 5-15. CP class is either A (a score of 5-6), B (7-9), or C (10 or above). Decompensation indicates cirrhosis with a CP score of 7 or more (Class B).
^{a}Grade 0: normal consciousness, personality, neurological examination, electroencephalogram.
Grade 1: restless, sleep disturbed, irritable/agitated, tremor, impaired handwriting, 5 cps waves.
Grade 2: lethargic, time-disoriented, inappropriate, asterixis, ataxia, slow triphasic waves.
Grade 3: somnolent, stuporous, place-disoriented, hyperactive reflexes, rigidity, slower waves.
Grade 4: unrousable coma, no personality/behavior, decerebrate, slow 2-3 cps delta activity.
Child-Pugh criteria: (Pugh RN, Murray-Lyon IM, Dawson JL, et al. Transection of the oesophagus for bleeding oesophageal varices. Br J Surg. 1973 Aug.; 60(8): 646-649Pugh 1973, Lucey MR, Brown KA, Everson GT, et al. Minimal criteria for placement of adults on the liver transplant waiting list: a report of a national conference organized by the American Society of Transplant Physicians and the American Association for the Study of Liver Diseases. Liver Transpl Surg. 1997 Nov.; 3(6): 628-637, Vilstrup H, Amodio P, Bajaj J, et al. Hepatic encephalopathy in chronic liver disease: 2014 Practice Guideline by the American Association for the Study of Liver Diseases and the European Association for the Study of the Liver. Hepatology. 2014 Aug.; 60(2): 715-735).

### Criteria for Assessing Cirrhosis

Acceptable methods of confirming (via medical history) or assessing cirrhosis include documented evidence or presence of one or more of the following cirrhosis indicators:
Biopsy results consistent with PBC Stage 4 (Ludwig J, Dickson E R, McDonald G S. Staging of chronic nonsuppurative destructive cholangitis (syndrome of primary biliary cirrhosis). Virchows Arch A Pathol Anat Histol. 1978;379(2):103112)
TE Median Value ≥16.9 kPa (Corpechot C, Carrat F, Poujol-Robert A, et al. Noninvasive elastography-based assessment of liver fibrosis progression and prognosis in primary biliary cirrhosis. Hepatology. 2012;56(1):198-208).
The presence of any of the following in the absence of acute liver failure:
   Varices
   Ascites
   Radiological evidence of cirrhosis (e.g. nodular liver or enlargement of portal vein and splenomegaly)
Combined low platelet count (<150,000/ml) with:
   persistent decrease in serum albumin, or
   elevation in PT/INR (not due to antithrombotic agent use), or
   elevated bilirubin (2× ULN)

### Mayo Risk Score

Mayo Risk Score (MRS) (Dickson E R, Grambsch P M, Fleming T R, et al. Prognosis in primary biliary cirrhosis: Model for decision making. Hepatology. 1989;10(1):1-7) is calculated and reported within the EDC system based on data entered into the eCRF. Calculation of MRS includes assessment of peripheral edema and the use of diuretic therapy, which is assessed during AE and concomitant medicine review at the scheduled visits and entered into the eCRF, as well as total bilirubin, albumin, and PT results obtained from the central laboratory data.

### Potential Myopathy Evaluation and Monitoring

Subjects are monitored for potential myopathy events throughout the study. If a subject becomes aware of the development of signs and symptoms of potential myopathy (i.e., muscle weakness, muscle soreness or muscle pain), the subject is instructed to promptly contact the site for further assessment.

Clinical criteria for the monitoring of these events and the interruption/discontinuation of IP are as follows:
At screening: Check baseline creatine kinase (CK): If >5× ULN then screen fail.
Previous History of Myalgia: Yes or No. If Yes, rate on a scale of 1 to 10 with 1 being the least amount of discomfort and 10 being the most amount of discomfort to establish baseline.
If subjects develop symptoms of myopathy at any point during the study, CPK values must be checked. A CPK≥5× ULN or ≥3× ULN with symptoms consistent with rhabdomyolysis will require IP interruption. Laboratory values including CK and creatinine will be closely monitored until values return to baseline levels.

### Medical Management of Subjects with Symptomatic Cholelithiasis and or Cholecystitis

If a subject experiences symptoms suggestive of cholelithiasis and/or cholecystitis, the subject undergoes a complete evaluation consistent with the local standard of care, is assessed for appropriate treatment, including potential indication for surgery (e.g., cholecystectomy), and is monitored until resolution of clinical signs and symptoms. If symptomatic cholelithiasis and/or cholecystitis is diagnosed, investigational product should be permanently discontinued.

recurrent bile duct stones and approval is received.

Investigational product should also be discontinued in the following instances:
Development of choledocholithiasis
Presence of any symptomatic gallbladder polyp; or
Presence of any gallbladder polyp>5 mm

### Dosage Adjustment Criteria

### Double Blind Phase

The dosage for OCA (5 mg) is maintained constant during the study. However, dose frequency may be modified for the management of pruritus or other safety findings.

In the event of tolerability issues such as pruritus or myalgia, the dosing frequency of all IP may be decreased.

Subjects can be discontinued from IP at any time for clinical safety concerns.

Subjects who are discontinued from IP in the DB Phase are scheduled for an end-of-double-blind (EODB) visit as close as possible to the last dose taken. Once the EODB visit has been completed the subject continues with their original scheduled visits until the end of the DB Phase where they complete the EODB visit and finish the study. These subjects will not participate in the LTSE portion of the study.

### LTSE Phase

Subjects who continue to meet protocol requirements transition to the LTSE Phase upon completion of the DB Phase. During the LTSE Phase, all subjects are maintained (Treatment D subjects already on transition dose) or transitioned to OCA 5 mg+400 mg BZF once daily.

In the LTSE, dose frequency may be modified for the management of pruritus or other safety findings. In the event of tolerability issues such as pruritus, the dosing frequency may be decreased.

Subjects can be discontinued from IP at any time for clinical safety concerns. Subjects who are discontinued from IP in the LTSE Phase should schedule for the end of study (EOS) Visit and complete the study.

### Overview of Assessments

| | |
|---|---|
| Analysis Variables | Endpoint Assessments |

Primary Objectives

| | |
|---|---|
| Change in ALP | Reduction in ALP from baseline to Week 12 |
| | in the DB Phase |

Secondary Objectives

| | |
|---|---|
| Safety and tolerability | SAEs, TEAEs, (including liver-related safety |
| | and DILI events) clinical laboratory |
| | assessments, physical examinations, ECGs, |
| | and vital signs, early discontinuations, and |
| | estimated glomerular filtration rate (eGFR) |
| Response and normalization rates of | ALP response rates of ≥10%, ≥20%, |
| biochemical disease markers | and ≥40% reduction from baseline at Week 12, |
| | and normalization at Week 12 |
| | Clinical laboratory values: change from |
| | baseline |
| | In ALP, GGT, ALT, AST, total and |
| | conjugated bilirubin; AST to platelet ratio |
| | index [(APRI) and lipid panel |
| Biomarkers of bile acid synthesis and | C4 and bile acids |
| homeostasis | |

Additional Objectives

| | |
|---|---|
| Noninvasive assessments of liver | Liver stiffness measured by TE, ELF score, |
| fibrosis | and markers of collagen formation and |
| | degradation (Pro-C3, Pro-C5, C3M, and C4M) |
| | as well as Fib4, and APRI |
| Assessment of clinical efficacy | ALP < 1.67x ULN and total bilirubin ≤ ULN |
| | and ALP decrease of ≥15% from baseline |
| Disease-specific symptoms as assessed | PBC-40, pruritus VAS, NRS, EQ-5D-5L, SF- |
| by Health-Related quality of life | 36 and Fatigue Impact Scale (FIS) |
| questionnaires | |
| Noninvasive assessment of liver | HepQuant SHUNT |
| function | |
| Disease severity scores | GLOBE, UK-PBC, MELD scores |
| PK | PK parameters for OCA, BZF and their |
| | metabolites |
| PK/PD relationships | Exposure vs PGx biomarkers and |
| | safety/efficacy markers |

ALP = alkaline phosphatase; ALT = alanine aminotransferase; APRI = AST to platelet ratio index; AST = aspartate aminotransferase; BZF = bezafibrate; C3M = type III collagen; C4 = 7α-hydroxy-4-cholesten-3-one; C4M = type IV collagen; ECG = electrocardiogram; eGFR = estimated glomerular filtration rate; ELF = enhanced liver stiffness; EQ-5D-5L = EuroQol 5-dimensional questionnaire; Fib4 = Fibrosis-4; FIS = Fatigue Impact Scale; GGT = gamma-glutamyl transferase; MELD = model of end-stage liver disease; OCA = obeticholic acid; PBC = primary biliary cholangitis; PD = pharmacodynamics; PGx = pharmacogenomics; PK = pharmacokinetic; Pro-C3 = type III pro-collagen; Pro-C5 = type V pro-collagen; SAE = serious adverse event; SF-36 = Short Form 36 Health Survey; TE = transient elastography; TEAE = treatment emergent- adverse event; UK = United Kingdom; VAS = visual analog scale.

### Efficacy Assessments

### Disease-Specific Symptoms as Assessed by Health-Related Quality of Life Questionnaires

Data is collected to determine the effects of OCA on health-related quality of life.

The following health-related quality of life measures are used to compare the effects of OCA alone or in combination with BZF:
PBC 40: A fully validated measure for use in PBC that has 6 domains (fatigue, emotional, social, cognitive function, general symptoms, and itch).
Pruritus VAS and NRS: Are assessments specifically to evaluate pruritus.
EQ-5D-5L: A standardized instrument for measuring generic health status that has 5 dimensions (mobility, self-care, usual activities, pain/discomfort, and anxiety/depression) and 5 levels for severity.
SF-36: A measure of health status that has 8 sections (vitality, physical functioning, bodily pain, general health perceptions, physical role functioning, emotional role functioning, social role functioning, and mental health).
Fatigue Impact Scale (FIS): The FIS is a validated 40 question questionnaire organized into 3 dimensions: cognitive, physical, and social. Each dimension has 5 levels: no problem, small problem, moderate problem, big problem, and extreme problem (Fisk J D, Ritvo P G, Ross L, et al. Measuring the functional impact of fatigue: initial validation of the fatigue impact scale. Clin Infect Dis. 1994 Jan;18 Suppl 1:S79-83).

### GLOBE and UK PBC Scores

The GLOBE and UK-PBC scores are calculated from serum chemistry and other parameters and will be recorded on the eCRF.

### Prospective Screening for Gallbladder Diseases

Screening for gallbladder disease consists of a detailed subject medical history and a baseline right upper quadrant ultrasound exam (if not performed within 3-month of the Screening Visit) to determine the presence of cholelithiasis and gallbladder disease. The diameter of the subject's common bile duct is recorded as well as an alpha-fetoprotein (AFP) assessment. For subjects who develop gallbladder disease during the study, gallbladder disease screening assessments for study visits after onset of gallbladder disease are not required.

### Analysis Populations

ITT Population: The Intent-to-Treat (ITT) Population includes all randomized subjects. Treatment assignment is based on the randomized treatment.

mITT Population: The modified ITT (mITT) Population includes all randomized subjects who have baseline and at least one post baseline ALP assessment. Treatment assignment is based on the randomized treatment.

Per-Protocol Population: The Per-Protocol Population includes all subjects in ITT Population without any major protocol deviations. Treatment assignment is based on the randomized treatment.

Safety Population: The Safety Population includes all randomized subjects who receive at least 1 dose of BZF and/or OCA. Treatment assignment is based on the actual treatment received.

Pharmacokinetic Population: The PK Population includes all subjects who receive BZF and/or OCA and have enough quantifiable PK samples without any major protocol deviations that could potentially affect plasma exposure.

LTSE Population: The LTSE Population includes all subjects who receive at least 1 dose of investigational product (IP)

### Determination of Sample Size

According to Corpechot C, Chazouillères O, Rousseau A, et al. A placebo-controlled trial of bezafibrate in primary biliary cholangitis. N Engl J Med. 2018;378:2171-2181, the median change from baseline in ALP at Month 24 of BZF 400 mg is −60 U/L with an interquartile range (IQR) from −66 U/L to −46 U/L based on 46 subjects. Since the IQR is approximately 1.35 times the standard deviation, the estimated standard deviation is approximately 15. The data from Month 24 is used in the sample size calculation with the assumption that ALP is stable from Month 3 to Month 24. In Study 747-301, the standard deviation of change from baseline of ALP at Month 3 is 74 based on 69 subjects. The pooled standard deviation of change from baseline at Month 3 based on those two treatments is 58. Assuming the change from baseline of ALP in BZF is −60 U/L, 15 subjects per arm will provide 80% power to detect a treatment difference of 60 U/L between BZF arm and BZF+OCA arm at two-sided alpha of 0.05 with the pooled standard deviation of 58.

### Primary Efficacy Analysis in Double-Blind Phase

The Primary Efficacy Analysis for the DB Phase, the change in ALP from baseline to Week 12, will be conducted using the mITT population when all subjects have completed the DB treatment period.

The intercurrent event in this study includes the use of prohibited medication and treatment discontinuation. For the analysis on the primary efficacy endpoint, data collected after the intercurrent events, such as the use of prohibited medications, and data collected after treatment discontinuation, is not included.

Analyses of change in ALP will be carried out using a mixed-effect repeated measures model (MMRM) to evaluate the effect of treatment groups over time. The dependent variable will be the change in ALP from baseline. The model will include the terms of treatment group, visit, treatment by visit interaction, and randomization stratification variables as factors and the baseline value as a covariate.

The primary efficacy analyses is also conducted in the Per-Protocol Population.

In addition, a sensitivity analysis using the mITT population is performed on the primary efficacy endpoint, where data collected after the intercurrent events is included in the analysis. This provides the evaluation of efficacy effect regardless of treatment adherence.

### Secondary and Additional Efficacy Analyses for the Double-Blind Phase

The ITT Population, e.g., all randomized subjects, are the primary population used for the secondary and additional efficacy analyses. The secondary and additional efficacy analyses are not analyzed in the Per-Protocol Population.

The secondary endpoints include:
The response rates of ≥10%, ≥20%, and ≥40% reduction from baseline at Week 12, and normalization rates at Week 12
Change from baseline of NRS, PBC-40, pruritus VAS, EQ-5D-5L, SF-36, and FIS at Week 12

The additional efficacy endpoints include:
Percent changes from baseline of ALP at Week 12
Change from baseline to Week 12 in GGT, ALT, AST, and total and conjugated bilirubin
Change from baseline to Week 12 in AST to platelet ratio index (APRI)
Normalization rates at Week 12 of GGt, ALT, AST, total and conjugated bilirubin
Change from baseline in noninvasive markers of liver fibrosis, including liver stiffness measured by TE, ELF score, Fib4, and markers of collagen formation and degradation (Pro-C3, ProC5, C3M, and C4M) at Week 12
Change from baseline in the GLOBE, UK-PBC, and MELD scores at Week 12
Percentage of subjects with ALP<1.67× upper limit of normal (ULN) and total bilirubin ≤ULN and ALP decrease of ≥15% from baseline at Week 12

For the secondary and additional efficacy endpoints, data collected after the intercurrent events, such as the use of prohibited medications, and data collected after treatment discontinuation are not included.

All continuous and categorical secondary and additional efficacy endpoints are summarized using descriptive statistics at baseline and at each scheduled post-baseline visit comparing the BZF and OCA+BZF treatment groups.

Analyses for ALP response rates of 10%, 20%, and 40% reduction from baseline at Week 12, and normalization rates and percentage of subjects with ALP<1.67× ULN and total bilirubin ≤ULN and ALP decrease of ≥15% from baseline are performed using a Cochran-Mantel-Haenszel test stratified by the randomization stratification factor.

Analyses of PBC-40, pruritus VAS, EQ-5D-5L, SF-36, NRS and FIS are performed using a MMRM model similar to that for the primary analysis.

Liver function evaluated by HepQuant-SHUNT is summarized with descriptive statistics at baseline and post-baseline visits.

### PK and PK PD Analyses for the Double-Blind Phase

PK analyses reported in the clinical study report (CSR) consist of individual subject listings and descriptive summary tables of concentrations of OCA and BZF and their metabolites. PK parameter analyses and PK/PD (i.e., exposure-response) analyses, including concentration-QTc modeling, are performed using a multiple-study/program-level approach to be described in a Pharmacometric Analysis Plan (PAP) and are reported in a Pharmacometric Analysis Report (PAR), separate from the CSR.

### LTSE Analyses

Similar analyses as those described for the DB Phase are conducted for the LTSE Phase using the DB baseline value, with the exception of PK, which is not performed during the LTSE Phase. Analyses based on the DB baseline are performed using randomized treatment groups in DB Phase.

### Interim Analysis

There is 1 planned interim analysis performed when at least 32 subjects complete the Week 12 visit in accordance with the SAP and DMC Charter. The primary endpoint for this planned interim analysis is the change from baseline in ALP at Week 12.

When all subjects have completed the DB Phase, the study converts to an LTSE.

The efficacy measures for the interim analysis include:
The change in ALP from baseline to Week 12 in the DB Phase in the mITT population
The ALP response rates of ≥10%, ≥20%, and ≥40% reduction from baseline at Week 12 and the normalization rates of ALP at Week 12 in the ITT population
Percentage of subjects with ALP<1.67× ULN, total bilirubin within normal limits, and ALP decrease of ≥15% in the DB Phase in the ITT population
The changes from baseline to Week 12 in GGT, ALT, AST, total bilirubin and conjugated bilirubin in the ITT population

In addition, the analyses of drug exposure and safety assessments will be performed.

Those endpoints will be summarized using descriptive statistics at baseline and at each scheduled post-baseline visit.

### Handling of Missing Data

Subjects who discontinue IP are expected to continue in the study until study termination. Missing data is assumed to be missing at random. No data is imputed.

### Safety Analyses

The Safety Population is the primary population used for safety analyses. Treatment assignment is based on the treatment actually received.

Safety data, including SAEs, TEAEs, physical examinations, ECGs, vital signs, clinical laboratory assessments, and treatment discontinuations, are compared across all treatment groups during the treatment phase.

The incidence of TEAEs and SAEs is tabulated by system organ class and preferred term for each treatment group and similarly by severity and relationship to treatment.

Laboratory parameters and vital signs are summarized (including Hy's law) by treatment group using descriptive statistics at baseline and at each scheduled postbaseline visit. The change from baseline will is summarized. ECGs are summarized by treatment group using frequency at each visit. The shift from baseline is be summarized. Baseline is defined as the mean of all available evaluations before treatment.

### Adverse Events

AEs are coded using the Medical Dictionary for Regulatory Activities (MedDRA). Summary tables of TEAEs will be provided. The incidence of TEAEs are tabulated by system organ class and preferred term for each treatment group and by severity and relationship to treatment. Summaries of TEAEs leading to IP discontinuation and SAEs are provided. Adverse events of special interest are summarized for each treatment group.

The incidence of TEAEs and SAEs is tabulated by system organ class and preferred term for each treatment group and similarly by severity and relationship to treatment.

### Clinical Laboratory Evaluations

Laboratory parameters and vital signs are summarized (including Hy's law) by treatment group using descriptive statistics at baseline and at each scheduled postbaseline visit. The change from baseline is also summarized. ECGs are summarized by treatment group using frequency at each visit. The shift from baseline is be summarized. Baseline is defined as the mean of all available evaluations before treatment (except for lipoprotein assessments where baseline will be Day 1).

In addition, shift tables from baseline based on normal ranges are provided for hematology, coagulation, and serum chemistry by treatment group to assess changes in laboratory values from baseline to each on-study evaluation.

### Example 3: HepQuant-SHUNT to Measure Liver Function to Assess to Assess Liver Disease and Treatment Effect

Hepatic inflammation and hepatic fibrosis impair hepatocyte function and hepatic perfusion. Evolution to cirrhosis is associated with increasing hepatic impairment-ultimately leading to portal hypertension and portal-systemic shunting. Portal hypertension (PH) is a risk factor for poor outcome in liver disease.

The HepQuant SHUNT test is an assay that is included as an additional study objective. This Example describes the HepQuant SHUNT test and its use for assessing liver disease and treatment effects in this study. The HepQuant tests measure the clearance of cholates labeled with molecular probes (carbon-13 [¹³C], and deuterium [4D]). In brief, the test involves placement of an indwelling peripheral venous catheter (usually in the antecubital vein of the arm), an injection of ¹³C-cholate (cold, stable label, NOT RADIOACTIVE) intravenously, and a drink of flavored solution of 40 mg d4-cholate (d4-CA or 4D-CA) (again, cold, stable label, NOT RADIOACTIVE). Blood samples will be taken at predose of cholate and 5, 20, 45, 60, 90 minutes post-dosing. Blood samples will be allowed to clot and be spun, and the serum will be transferred to transport tubes for mailing to HepQuant lab for processing and analysis. HepQuant SHUNT tests are capable of monitoring hepatocellular function, total hepatic perfusion, portal inflow to the liver, and portal-systemic shunting. Similar to HVPG, HepQuant SHUNT assesses the portal circulation, but is non-invasive with high subject tolerability and lower cost.

Liver diseases alter hepatocyte function and the portal circulation which manifests as portal hypertension and portal-systemic shunting. The clinical consequences are coagulopathy, jaundice, varices, ascites and encephalopathy. As liver disease progresses, from early stage with minimal fibrosis to late stage fibrosis, cirrhosis, and clinical complications, hepatic function and the two circulatory inflows to the liver, systemic and portal, become increasingly impaired. The HepQuant-SHUNT test measures a liver-specific function, clearance of cholate, from both systemic and portal circulations simultaneously. The test is based on the fact that liver disease impairs function and alters the portal circulation. As blood flow to the liver becomes impaired, a greater amount of the administered cholates escapes extraction by the liver and spills over into the systemic circulation; this is manifested as an increase in systemic cholate concentration in the blood samples obtained through the peripheral venous catheter. HepQuant SHUNT quantifies the changes in liver function and the portal circulation from early through late stages of disease.

In prior studies of chronic hepatitis C, the Disease Severity Index (DSI) from the HepQuant SHUNT test correlated with ISHAK and METAVIR stages of fibrosis and predicted likelihood of cirrhosis, varices, and risk for clinical outcome. DSI has performed similarly in patients with chronic hepatitis C, NAFLD, and PSC. The HepQuant study is to compare the change in DSI between treatment arms at each time point over the time period of the study.

### Test Administration

The HepQuant SHUNT test is performed after at least 5 hours of fasting, usually after an overnight fast, and requires venous access via a standard indwelling peripheral venous catheter, preferably placed in the antecubital fossae. Approximately 3 mL blood samples are obtained at baseline and at 5, 20, 45, 60, and 90 minutes after dosing with the cholate solutions; and, ≥1 mL serum is shipped at ambient temperature to the HepQuant laboratory for analysis of cholate concentrations. The subject may be in a bed seated upright or in a recliner chair-the subject should be seated in an upright position, or if in bed, have the head of the bed elevated by at least 30 degrees to aid gastric emptying of the orally administered dose of 4D-CA solution.

Prior to administration, the HepQuant SHUNT Liver Diagnostic Kits are kept at ambient temperature. For the oral 4D-CA dose, the full contents of the d4-CA solution are poured into a 40 mL cup and flavoring added. For the intravenous ¹³C-CA dose, 5 mL (from a total of 5.5 mL) are removed from the ¹³C-CA solution vial and mixed with 5 ml of the albumin solution (25% w/v human serum albumin, USP grade, GRIFOLS). The ¹³C-CA/Albumin mixture is injected intravenously over 1 minute by the person administering the test. The 4D-cholate/flavoring mixture is administered orally simultaneously over the same minute.

The test can be administered in one of two methods: (1) the Two-Arm method and (2) the Single-Arm, Single-Catheter method. The Two-Arm method uses the intravenous (IV) catheter solely for blood sampling. A separate butterfly or small catheter, placed in the opposite arm, is used for injection of the IV cholate/albumin solution. The Two-Arm method is the preferred method of administration. The Single-Arm, Single-Catheter method uses the same catheter for both injection of the IV cholate/albumin and subsequent blood sampling. A strict flushing procedure should be used if the Single-Arm, Single-Catheter is used—to avoid carryover of the injected cholate solution into the subsequent blood samples. If the subject were to experience an anaphylactic or hypersensitivity reaction to the compounds, administration should be stopped, and the subject should be treated in accordance with standard of care. The subject should not undergo any future HepQuant tests, but may remain enrolled in the parallel drug study.

### Test Outputs

Cholate concentrations (endogenous unlabeled CA, ¹³C-CA, and d4-CA) will be measured from the timed serum samples (0, 5, 20, 45, 60, and 90 minutes) and concentrations of each labeled cholate as a function of time will be modeled as a spline curve in order to calculate the area under curve (AUC). The HepQuant SHUNT test parameters are:
Systemic HFR: The intravenous clearance (Clᵢᵥ, mL min⁻¹) is defined as the dose/AUC for ¹³C-CA. The Systemic HFR is defined as the Clᵢᵥ per kg of body weight and is expressed as mL min⁻¹ kg⁻¹.
Portal HFR: The apparent oral clearance (Clₒᵣₐₗ, mL min⁻¹) is defined as the dose/AUC for d4-cholate. The Portal HFR is defined as the Clₒᵣₐₗ per kg of body weight and is also expressed as mL min−1 kg−1.
SHUNT: SHUNT, the portal-systemic shunt fraction, is calculated as the ratio Systemic HFR/Portal HFR×100%.
DSI: The calculation for Disease Severity Index is according to a formula derived from Systemic HFR and Portal HFR.
STAT: The serum concentration of d4-cholate from the 60-minute blood sample correlates well with DSI (r2=0.88) and is independently analyzed for links to disease severity and treatment effects, similar to the analyses for DSI.

### Known Potential Risks

The risks associated with the test compounds include: (1) Allergic reaction to cholate compounds (theoretical—none yet reported); and (2) Allergic reaction to human serum albumin (HSA), where reactions could include: (a) rash; (b) having a hard time breathing; (c) wheezing when you breathe; (d) sudden drop in blood pressure; (e) swelling around the mouth, throat, or eyes; (f) fast pulse; (g) sweating; (h) severe reactions are very rare but a severe reaction (called anaphylaxis); and (i) can lead to profoundly low blood pressure and even death.

The risks associated with the indwelling catheter include: (1) Pain with placement of catheter; (2) Thrombosed vein; and (3) Hematoma.

The risks associated with phlebotomy include: (1) Localized pain; (2) Bruising; (3) Occasional lightheadedness; (4) Fainting; and (5) Infection at the site (rare).

The risks associated with fasting include: (1) Dizziness; (2) Headache; (3) Stomach Discomfort; and (4) Fainting.

### Test Compounds

Cholates, labeled with stable (nonradioactive) isotopes, occur naturally and are not known to have any deleterious or adverse effects when given intravenously or orally in the doses used in HepQuant (HQ) tests. The serum cholate concentrations that are achieved by either the intravenous or oral doses are similar to the serum concentrations of bile acids that occur after the ingestion of a fatty meal.

The two cholates used in the HepQuant test for this study are labeled with stable (non-radioactive) forms of carbon and hydrogen that are found in nature and can be measured in blood. These forms of cholate have been used with FDA INDs (65121 and 65123) since 2002, and their use in humans has been monitored since that time.

### Analysis of Results

Liver function evaluated by HepQuant-SHUNT will be summarized with descriptive statistics at baseline and post-baseline visits. The major objective of this HepQuant SHUNT study is to determine whether serial changes in DSI indicate a treatment effect, and to define the relationship of change in DSI to change in other measures of treatment response. Further analysis details will be specified in the SAP and/or a separate clinical pharmacology analysis plan. For responder analyses using DSI as the endpoint. a significant treatment response in a given subject will be defined as a two point or greater decrease in DSI.

1. A method of treating or preventing an FXR mediated disease or condition in a subject in need thereof, comprising administering to the subject obeticholic acid (OCA) or a pharmaceutically acceptable salt, solvate, or amino acid, sulfate or glucuronide conjugate, or prodrug thereof in an amount of between about 5 and about 10 mg, and bezafibrate or a pharmaceutically acceptable salt or ester thereof in an amount between about 100 mg and about 400 mg.

2. The method of claim 1, wherein the obeticholic acid (OCA) or pharmaceutically acceptable salt, solvate, or amino acid, sulfate or glucuronide conjugate, or prodrug thereof is administered in an amount of about 5 mg.

3. The method of claim 1, wherein the obeticholic acid (OCA) or pharmaceutically acceptable salt, solvate, or amino acid, sulfate or glucuronide conjugate, or prodrug thereof is administered in an amount of about 10 mg.

4. The method of claim 1, wherein the bezafibrate or pharmaceutically acceptable salt or ester thereof is administered in an amount of about 100 mg.

5. The method of claim 1, wherein the bezafibrate or pharmaceutically acceptable salt or ester thereof is administered in an amount of about 400 mg.

6. The method of claim 1, comprising administering to the subject an effective amount of OCA.

7. The method of claim 1, comprising administering to the subject an effective amount of bezafibrate.

8. A method of treating or preventing an FXR mediated disease or condition in a subject in need thereof, comprising administering to the subject a first pharmaceutical composition comprising obeticholic acid (OCA) or a pharmaceutically acceptable salt, solvate, or amino acid, sulfate or glucuronide conjugate, or prodrug thereof in an amount of between about 5 and about 10 mg, and a second pharmaceutical composition comprising bezafibrate or a pharmaceutically acceptable salt or ester thereof in an amount between about 100 mg and about 400 mg.

9. The method of claim 8, wherein the first pharmaceutical composition comprises OCA.

10. The method of claim 9, wherein the second pharmaceutical composition comprises bezafibrate.

11. The method of claim 8, wherein the first pharmaceutical composition comprises OCA or pharmaceutically acceptable salt, solvate, or amino acid, sulfate or glucuronide conjugate, or prodrug thereof in an amount of about 5 mg.

12. The method of claim 8, wherein the first pharmaceutical composition comprises OCA or pharmaceutically acceptable salt, solvate, or amino acid, sulfate or glucuronide conjugate, or prodrug thereof in an amount of about 10 mg.

13. The method of claim 8, wherein the second pharmaceutical composition comprises bezafibrate or pharmaceutically acceptable salt or ester thereof in an amount of about 100 mg.

14. The method of claim 8, wherein the second pharmaceutical composition comprises bezafibrate or pharmaceutically acceptable salt or ester thereof in an amount of about 400 mg.

15. The method of claim 8, wherein the first pharmaceutical composition is a tablet.

16. The method of claim 8, wherein the second pharmaceutical composition is a tablet.

17. The method of claim 8, wherein the first pharmaceutical composition is an immediate release form.

18. The method of any claim 8, wherein the first pharmaceutical composition is a sustained release tablet.

19. The method of any one of claim 8, wherein the second pharmaceutical composition is an immediate release form.

20. The method of claim 8, wherein the second pharmaceutical composition is a sustained release tablet.

21. The method of claim 1, wherein the FXR mediated disease or condition is selected from primary biliary cholangitis (PBC), primary sclerosing cholangitis (PSC), portal hypertension, bile acid diarrhea, a chronic liver disease, nonalcoholic fatty liver disease (NAFLD), nonalcoholic steatohepatitis (NASH), hepatitis C infection, an alcoholic liver disease, liver damage due to progressive fibrosis, liver fibrosis, drug-induced cholestasis, hereditary cholestasis, biliary atresia, and intrahepatic cholestasis of pregnancy.

22. The method of claim 21, wherein the FXR mediated disease or condition is chronic liver disease.

23. The method of claim 21, wherein the FXR mediated disease or condition is a cholestatic liver disease.

24. The method of claim 21, wherein the FXR mediated disease or condition is PBC.

25. The method of claim 21, wherein the FXR mediated disease or condition is NASH.

26. The method of claim 21, wherein the FXR mediated disease or condition is liver fibrosis.

27. The method of claim 21, wherein the FXR mediated disease or condition is liver fibrosis associated with NASH.

28. The method of claim 1, wherein the OCA or pharmaceutically acceptable salt, solvate, or amino acid, sulfate or glucuronide conjugate, or prodrug thereof and the bezafibrate or pharmaceutically acceptable salt or ester thereof are administered sequentially.

29. The method of claim 1, wherein the OCA or pharmaceutically acceptable salt, solvate, or amino acid, sulfate or glucuronide conjugate, or prodrug thereof and the bezafibrate or pharmaceutically acceptable salt or ester thereof are administered concurrently.

30. The method of claim 1, wherein the OCA or pharmaceutically acceptable salt, solvate, or amino acid, sulfate or glucuronide conjugate, or prodrug thereof is administered prior to the administration of the bezafibrate or pharmaceutically acceptable salt or ester thereof.

31. The method of claim 1, wherein the OCA or pharmaceutically acceptable salt, solvate, or amino acid, sulfate or glucuronide conjugate, or prodrug thereof is administered subsequent to the administration of the bezafibrate or pharmaceutically acceptable salt or ester thereof.

32. (canceled)

33. A pharmaceutical composition comprising OCA and a pharmaceutically acceptable carrier or excipient for use in a method according to claim 1.

34. (canceled)

35. (canceled)

36. (canceled)
